(19) United States
(12) Patent Application Publication
(10) Pub. No.: US 2011/0053859 A1
(43) Pub. Date: Mar. 3, 2011

(54) METHODS TO REDUCE THE EFFECTS OF SLEEP DEPRIVATION

(75) Inventors: Samuel A. Deadwyler, Lewisville, NC (US); Robert E. Hampson, Kernersville, NC (US); Linda Porrino, Lewisville, NC (US); Michael Todd, Winston Salem, NC (US); Thomas N. Thannickal, Winnetka, CA (US); Yuan-Yan Lai, North Hills, CA (US); Jerome M. Siegel, Northridge, CA (US)

(73) Assignee: The U. S. Government Respresented by the Department of Veterans Affairs, Washington, DC (US)

(21) Appl. No.: 12/682,563

(22) PCT Filed: Oct. 10, 2008
(86) PCT No.: PCT/US08/79591

§ 371 Date: Nov. 16, 2010

Related U.S. Application Data
(60) Provisional application No. 60/978,979, filed on Oct. 10, 2007.
(60) Provisional application No. 61/041,798, filed on Apr. 2, 2008.

(51) Int. Cl. A61K 38/17; A61P 25/00; A61P 25/16; C12Q 1/02

(52) U.S. Cl. 514/18.1; 435/29

(57) ABSTRACT

The invention provides methods of treating symptoms of sleep deprivation using a hypocretin agonist. The invention also provides methods of treating Parkinson's disease using a hypocretin agonist.

### CROSS-REFERENCES TO RELATED APPLICATIONS

The present application is a nonprovisional of and claims the benefit of 61/041,798 filed Apr. 2, 2008 and 60/978,979 filed Oct. 10, 2007, both of which are incorporated by reference in their entirety for all purposes.

### STATEMENT AS TO RIGHTS TO INVENTIONS MADE UNDER FEDERALLY SPONSORED RESEARCH OR DEVELOPMENT

This research was supported by DARPA DAAD19-02-1-0060 and NIH Award Nos. DA000119, DA006634, DA009085, MH064109, NS014610, HL041370, and NS042566. The government has certain rights to this invention.

### BACKGROUND OF THE INVENTION

The invention relates to methods to reduce the effects of sleep deprivation.

Orexin-A (hypocretin-1) is a potent sleep related peptide, secreted by specific neurons in the HPA axis (Peyron, 1998, van den Pol, 1998, Horvath, 1999, Moore, 2000, Lee, 2005). Receptors for orexin-A are located on neurons in many different brain regions, making it possible for this peptide, once released, to affect a large number of areas involved in sleep and sleep deprivation (Hagan, 1999, Bourgin 2000, Kilduff, 2000, Piper, 2000. Gerashchenko, 2001, Yoshida, 2001, Peever, 2002, Wu, 2002, Lee, 2005, Mileykovskiy, 2005, Vittoz, 2006).

It has recently been shown that manipulation of this system via administration of a hypocretin-1 antagonist can regulate sleep-waking in rats, dogs and humans (Brisbare-Roch 2007). When administered centrally in animal models, orexin-A has been shown to produce instant arousal and increased attention (Hagan 1999, Siegel, 2004, Mileykovskiy, 2005, White 2005, Borgland 2006). In addition, orexin-A has been implicated in counteracting the effects of sleep disorders such as narcolepsy in animal models (Chemellii 1999, Lin 1999, Wu, 2002, Gerashchenko, 2001, Mieda, 2004) as well as in humans (Nishino, 2000, Peyron, 2000, Thannickal, 2000).

Sleep deprivation in humans is a significant problem. Sleep deprivation affects shift workers, parents of newborns, long-distance drivers, personnel whose jobs require extended periods of wakefulness, as well as people suffering from chronic sleep deprivation due to pain, illness, insomnia, sleep apnea, etc.

Symptoms of sleep deprivation are similar to those of chronic stress. Sleep deprivation interferes with the behavioral performance of a variety of tasks, including cognitive, motor, attention, and motivation. The human body requires 6-9 hours of sleep per day for optimum cognitive function. A total or partial loss of sleep impairs the ability to correctly process information and make appropriate decisions. See also U.S. Patent Application Publication 2006/0276462 to Deadwyler Therefore there is a need to provide methods to alleviate the symptoms associated with sleep deprivation.

### BRIEF SUMMARY OF THE INVENTION

Provided herein are methods of treating or preventing at least one symptom of sleep deprivation (e.g., cognitive impairment) in a subject in need thereof, including administering a hypocretin agonist to the subject. In some embodiments, the hypocretin agonist is orexin-A, an analog thereof, a prodrug thereof, or a pharmaceutically acceptable salt of any thereof, in an amount effective to treat or prevent the symptoms. In some embodiments, administering is carried out by intranasal administration. In some embodiments, the subject is a human selected from the group consisting of: a pilot, a soldier, a law enforcement officer, a health care worker, a caretaker, a shift worker, and a person who voluntarily extends their waking period, wherein said subject would suffer from one or more symptoms of sleep deprivation in the absence of said administering.

Methods of coadministration of a hypocretin agonist and at least one additional active compound (e.g., caffeine, nicotine, amphetamine, Modafinil, or an AMPA receptor potentiator compound such as CX717) are also provided.

Compositions including a hypocretin agonist in a pharmaceutically acceptable carrier in combination with at least one additional active compound as described above are also provided.

Devices for intranasal administration including a hypocretin agonist (e.g., orexin-A) in a pharmaceutically acceptable carrier are further provided. In some embodiments, the device includes a glass or plastic container (e.g., a plastic squeeze bottle).

Also provided is the use of a hypocretin agonist for the preparation of a medicament for carrying out a method as described above for the treatment or prevention of symptoms of sleep deprivation.

The invention provides a method of treating at least one symptom of sleep deprivation in a subject in need thereof, comprising administering a hypocretin agonist to said subject in an amount effective to treat said at least one symptom of sleep deprivation. Optionally, the agonist is orexin-A, an analog thereof, a prodrug thereof, or a pharmaceutically acceptable salt of any thereof, in an amount effective to treat or prevent said at least one symptom of sleep deprivation. Optionally, the administering is carried out by intranasal administration. Optionally, the intranasal administration is by a nasal spray. Optionally, the administration is performed within an hour before performing or during performance of a task whose performance would otherwise be impaired by the sleep deprivation. In some methods, the task requires a high cognitive load. Optionally, the dose is 0.1-10 μg or 0.1-2 μg. In some methods, the administration is performed at irregular intervals responsive to the patient performing tasks whose performance would otherwise be impaired by the sleep deprivation. Optionally, the administration is performed multiple times during a continuous wake phase. Optionally, the task requires a high cognitive load. Optionally, the subject lacks a diagnosed sleep disorder or known biochemical or genetic marker of a sleep disorder. Optionally, the subject is free of narcolepsy, REM sleep behavior disorder, period leg movements in sleep and restless leg syndrome, circadian rhythm disorder, sleep apnea, hypersomnia, insomnia, Alzheimer's disease, depression, schizophrenia and obesity. Optionally, the subject is not in need of consolidation of sleep and waking states. Optionally, the subject is free of narcolepsy and cataplexy. Optionally, the administration does not promote greater consolidation of sleep and waking states in the patient. Optionally, the administration occurs after at least 12 hours of a wake phase. Optionally, the administration occurs after at least 24 hours of a wake phase. Optionally, the hypocretin agonist is carried by a pharmaceutically acceptable solid carrier. Optionally, the hypocretin agonist is carried by a pharmaceutically acceptable liquid carrier. Optionally, the symptom comprises cognitive impairment. Optionally, the administration improves the cognitive performance of the subject above a level without sleep deprivation. Optionally, the subject is human. Optionally, the subject is an adult.

Some methods also entail administering at least one additional active compound for treating at least one symptom of sleep deprivation to the subject, optionally, selected from the group consisting of: caffeine, nicotine, amphetamines, Modafinil and AMPA receptor potentiators. Optionally, the additional active agent is CX717.

The invention further provides for the use of a hypocretin agonist for the preparation of a medicament for carrying out a method according to any preceding claim. The invention further provides a hypocretin agonist for use in treating cognitive impairment due to sleep deprivation. Optionally, the hypocretin agonist of claim 29 formulated for nasal administration, optionally as a spray.

The invention further provides a composition comprising, in combination in a pharmaceutically acceptable carrier: (a) a hypocretin agonist; and (b) at least one additional active compound for treating at least one symptom of sleep deprivation. Optionally, the hypocretin agonist is orexin-A, an analog thereof, a prodrug thereof, or a pharmaceutically acceptable salt of any thereof, in an amount effective to treat or prevent said at least one symptom of sleep deprivation. Optionally, the at least one additional active compound is selected from the group consisting of: caffeine, nicotine, amphetamine, Modafinil and AMPA receptor potentiators. Optionally, the at least on additional active compound is CX717.

The invention further provides in a device for intranasal administration comprising an intranasal delivery container and a pharmaceutical formulation in said container, said pharmaceutical formulation comprising an active agent in a pharmaceutically acceptable carrier, the improvement comprising: using a hypocretin agonist as said active agent. Optionally, the device comprises a glass or plastic container. Optionally, the container comprises a plastic squeeze bottle. Optionally, the hypocretin agonist is orexin-A. Optionally, the hypocretin agonist further comprises a solid carrier. Optionally, the hypocretin agonist further comprises a liquid carrier.

The invention further provides a method of treating a symptom of Parkinson's disease comprising administering hypocretin or a hypocretin analog to a patient having Parkinson's disease. In some methods, the symptom is daytime sleep attacks, nocturnal insomnia, a REM sleep behavior disorder, a hallucination or depression. Optionally, the methods further entail administering a dopamine agonist, a serotonin selective reuptake inhibitor, or a norepinephrine reuptake inhibitor. In some methods, the hypocretin or analog is administered by nasal administration. In some methods, the dose is 0.1 to 10 μg orexin-A administered nasally. In some methods, the dose is administered daily.

The invention further provides methods of determining a clinical stage of Parkinson's disease in a patient, comprising: determining a level of hypocretin in a body fluid of the patient, wherein loss of hypocretin is correlated with clinical stage of the disease.

### BRIEF DESCRIPTION OF THE DRAWINGS

FIG. 1: Effects of IV orexin-A on DMS performance in alert monkeys. Three different dose levels (2.5, 5.0 and 10.0 μg/kg) of orexin-A were administered to alert non sleep deprived monkeys at the normal daily testing time. Sessions in which saline was administered in the same manner are plotted for comparison. Overall performance is shown as mean±SEM for all trials (gray bars) and for low (white) and high (black) cognitive load trials. Horizontal line=mean over all saline trials. Asterisks indicate *p<0.01, **p<0.001 relative to similar trial types in saline sessions.

FIG. 2: Effects of IV orexin-A on DMS task performance administered to sleep deprived monkeys. Sleep deprivation for 30-36 hrs significantly impaired DMS performance (SD, saline) compared to normal alert test sessions (Alert, saline). IV orexin-A (SD+orexin-A, 5.0, 10.0 μg/kg) injected 10-15 min prior to testing in sleep deprived animals partially reversed the detrimental effects on performance. Trials sorted according to cognitive load as in FIG. 1. Horizontal line=mean over all trials in normal alert saline sessions. Asterisks indicate **p<0.001 relative to similar trial types in non-sleep deprived saline sessions; †p<0.01 ‡p<0.001 indicate comparisons between IV orexin-A at different doses and sleep deprived (saline) sessions.

FIG. 3: Illustration of nasal application method of orexin-A to monkeys via atomizer spray. Atomizer shown at left was powered by compressed air and pulsed by computer controlled pressure valve for 50 ms spray duration. The applicator tip of the atomizer was positioned to within 5-8 cm of the nostrils of the chaired monkey and the pulsed orexin-A spray mist directed toward the midpoint between both nostrils. Two 50 ms sprays separated by 3-5 sec were delivered 5-10 min prior to start of the DMS testing session. Sprays of sterile water or saline on were delivered on nondrug testing sessions through the same atomizer system. The concentration and estimated volume of the nasal orexin-A spray mist are listed.

FIG. 4: Effects of nasal orexin-A (1.0 μg) on DMS performance of alert and sleep deprived monkeys. Left: Alert-There were no significant effects of nasal orexin-A on mean correct (±SEM) performance overall trials, or on low or high cognitive load trials in alert (nonsleep deprived) monkeys. Right: Sleep Deprived (SD)-Application of nasal orexin-A reversed the detrimental effects of 30-36 hr sleep deprivation (**p<0.001=comparison to saline alert) by significantly improving overall mean % correct (±SEM) performance as well as performance on low and high cognitive load trials (†p<0.01, ‡p<0.001=comparison to sleep deprivation).

FIG. 5: Effects of nasal orexin-A (1.0 μg) on performance of extended delay trials in DMS task. Data in FIG. 4 are re-plotted with mean % correct (±SEM) performance on DMS trials of more difficult (i.e. extended) delays (90-120 sec). Performance on these trials was significantly reduced (##F(_{1,503})>32.91, p<0.001) under all conditions (normal alert, sleep deprived and orexin-A sleep deprived) relative to low cognitive load trials. Under sleep deprived conditions performance on extended delay trials was significantly improved (‡F(_{1,503})=26.30, p<0.001) by delivery of nasal orexin-A relative to overall and high cognitive load trial comparisons in sleep deprived saline sessions.

FIG. 6: Comparison of IV (left) and nasal (right) orexin-A on reversal of the effects of sleep deprivation (SD) on DMS performance. Same data as in FIGS. 1 and 4 plotted to show comparison of effects of two delivery methods on mean % correct (±SEM) performance relative to very similar baseline (saline) sleep deprivation (SD) performance levels (**p<0.001=differences from sleep deprivation condition (†p<0.01, ‡p<0.001=differences between orexin-A delivery methods (IV vs. nasal) in sleep deprived animals).

FIG. 7: orexin-A reverses the effects of sleep deprivation on DMS task-related brain local CMRglc. Each column shows difference images of PET scans taken in the two different experimental conditions indicated at the top. Scaled color changes (color bar: far right) indicate magnitude of local CMRglc during performance of DMS task as determined from SPM constructed voxel maps at three different rosto-caudal brain levels to illustrate changes in specific brain regions engaged by the task. Left: Local CMRglc in the indicated brain regions was differentially altered by sleep deprivation in comparison to alert sessions. Middle: Administration of IV orexin-A (10.0 μg/kg) to the same monkeys that were sleep deprived and tested in the same manner. Increased local CMRglc relative to sleep deprived saline sessions in DSLFC, Str, and Thal and decreased CMRglc in MTL correspond to changes in DMS task performance noted in FIG. 2. Right: Administration of nasal orexin-A (1.0 μg) to sleep deprived monkeys produced marked increases in local CMRglc relative to sleep deprived saline sessions in: DSLFC, Str, and Thal and as well as decreased CMRglc in MTL. Color scale at right depicts degree of increase (yellow to red) or decrease (light blue to dark blue) in local CMRglc relative to comparison image. Abbreviations: dorsolateral prefrontal cortex, striatum: thalamus: medial temporal lobe: MTL.

FIG. 8. Distribution of Hcrt cells in normal and across PD stages. The clinical stages of PD are based on Hoehn and Yahr criteria. The cell distribution and count from a section of anterior, middle and posterior part of the hypothalamus were mapped from a normal, stage III and stage V of PD brains. The cell counts are listed for each section. The number of Hcrt cells is decreased with severity of the disease. 3v—third ventricle, Fx—fornix, Mmb—mammilary body, Opt—optic tract. Scale bars—50 μm.

FIG. 9: Distribution of MCH cells in normal and Parkinson stages. Cell counts are listed in each section. The number of MCH cell was decreased with severity of the disease. The abbreviations are same as in FIG. 1. Scale bars—50 μm.

FIG. 10: Hcrt and MCH pathology in different stages of PD. A, the total number of Hcrt and MCH cells in normal and PD-I, PD-II, PD-III, PD-IV and PD-V. The values are compared to cell numbers in the normal brains. B, the size of the Hcrt, MCH and neuromelanin pigmented cells estimated by nucleator method. Hcrt and MCH cells in PD did not differ in size from those in normal brains. Neuromelanin pigmented cells showed hypertrophy (27%) compared with normal cells. C, Hcrt and MCH cells were mapped in individual sections from anterior to posterior hypothalamus with 1200 μm section interval. One brain from a normal and one from each stage (Hoehn & Yahr, I-V) of PD were used for Neurolucida mapping. There was a generalized loss of Hcrt and MCH cells with severity of the disease. *p<0.05, **p<0.01, ***p<0.001, ****p<0.0001, Student's t test.

FIGS. 11A-F: Distribution of alpha synuclein in the hypothalamus in different stages of PD. A, Neurolucida mapping of alpha synuclein in PD stages with single immunostaining. B, mapping of Hcrt and alpha synuclein in double labeled section. C, mapping of MCH and alpha synuclein in double labeled section. Alpha synuclein was not colocalized with Hcrt and MCH cells (D & E), but it was colocalized with neuromelanin pigmented cells in substantia nigra (F). Arrows: red—alpha synuclein, green—Hcrt cell, black—MCH cells, and blue—neuromelanin pigmented cell. Scale bars—50 μm.

FIG. 12: Gliosis and neuromelanin pigmented cell loss in PD. A, the percentage loss of neuromelanin pigmented cell loss in the substantia nigra was correlated with duration of the disease. B, the number of glial fibrillary acidic protein-labeled astrocytes (GFAP) in the thalamus and posterior hypothalamus. C, GFAP in the hypothalamus of normal (a) and PD (b). GFAP density in the substantia nigra of normal (c) and PD (d) brain. The number of GFAP labeled astrocytes were increased with severity of the disease. Scale bars—50 μm.

FIG. 13: Nasal administration of hyocretin has no effect on cognitive performance when administered to alert monkeys, but reverses cognitive deficits in drowsy monkeys near the end of their normal day.

### DETAILED DESCRIPTION OF THE INVENTION

The present invention is explained in greater detail below. This description is not intended to be a detailed catalog of all the different ways in which the invention may be implemented, or all of the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

As used in the description of the invention and the appended claims, the singular forms “a”, “an” and “the” are intended to include the plural forms as well, unless the context clearly indicates otherwise. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” refers to and encompasses any and all possible combinations of one or more of the associated listed items, as well as the lack of combinations when interpreted in the alternative (“or”).

The disclosures of all United States patent references, other publications, Genbank citations and the like cited herein are hereby incorporated by reference for all purposes to the extent they are consistent with the disclosures herein. If a Genbank citation is associated with more than variant of a sequence, the variant of the sequence assigned the GenBank ID No. at the filing date of the application or priority application (if disclosed in the priority application) is meant. Unless otherwise apparent from the context, any embodiment, aspect, feature or step can be used in combination with any other.

It has been discovered that orexin-A significantly improved cognitive performance in primates in a dose-dependent manner, and reduced the effects of sleep deprivation.

### A. Definitions.

“Sleep deprivation” occurs when a subject fails to get the amount and/or quality of sleep required to function without the onset of one or more symptoms associated with sleep deprivation. For example, it is recommended that human subjects get 7-8 hours of sleep per night, though the optimum amount among individuals may vary. Sleep deprivation may be “continuous” or “acute” when lasting for a relatively short time, e.g., only for 1-3 sleep cycles. For example, a subject may be awake continuously for 24-72 hours. Sleep deprivation may also be chronic, normally including inadequate sleep across 4 or more sleep cycles, and thereby incurring a “sleep debt.” A “sleep cycle” normally includes an awake period, sleep stages 1, 2, 3, 4 and REM sleep.

“Symptoms” associated with sleep deprivation include, but are not limited to, deleterious effects on behavioral performance of a variety of tasks, including a decline in cognitive performance, a decline in motor performance, a decrease in attention span and/or attentiveness, decreased motivation, increased lethargy, etc., including combinations thereof. For example, sleep deprivation is commonly associated with cognitive impairment. Cognitive impairment can be measured by, e.g., established tests such as the multi-image delayed match to sample (DMS) short-term memory task (see, e.g., Porrino 2005).

“Treat” or “treating” herein refers to any type of treatment or prevention that imparts a benefit to a subject afflicted with or at risk of developing symptoms associated with sleep deprivation, including improvement in the condition of the subject (i.e., relief of one or more symptoms), delay or slowing of the progression symptoms, or of the severity of symptoms, etc. As such, the term “treatment” also includes prophylactic treatment of the subject to prevent the onset of symptoms. As used herein, “treatment” and “prevention” are not necessarily meant to imply a cure or complete abolition of symptoms.

“Treatment effective amount” as used herein means an amount of the active compound sufficient to produce a desirable effect upon a patient inflicted with the condition being treated, including improvement in the condition of the patient (e.g., in one or more symptoms), delay in the progression of the disease, etc.

“Subjects” in need of treatment by the methods described herein include subjects afflicted with or at risk for suffering from symptoms associated with sleep deprivation. Subjects may be male or female and at any stage of development, including, but not limited to, neonate, infant, juvenile, adolescent, young adult, adult, and geriatric subjects. While the present invention is concerned primarily with the treatment of human subjects, the invention may also be used for the treatment of animal subjects, particularly mammalian subjects such as dogs, cats, horses, cows, pigs, etc., for, e.g., veterinary purposes.

Subjects contemplated as benefiting from the practice of the present invention include, but are not limited to, persons or other mammals with circadian rhythm disruption such as, but not limited to: shift workers who must alter their activities from day to night or vice-versa, and hence encounter sleep loss and suffer associated symptoms due to the disruption of the sleep cycle; persons on extended work assignments, such as pilots, military personnel, law enforcement officers, truckers, health care workers or service animals, for whom continual alertness (and consequent loss of sleep) is essential to their task or their personal safety; persons who travel quickly through multiple time zones and must perform cognitive tasks before they are fully adjusted to the new zone (jet-lag); caretakers of newborns/invalids/critically ill patients, who typically must awaken frequently during the night to care for another person; patients with disease states that disrupt sleep, such as insomnia, sleep apnea, chronic pain, etc.; and persons who voluntarily extend their waking period beyond normal limits such that the resulting loss of sleep causes a cognitive decline; persons who have temporarily lost sleep through excessive drinking of alcohol, or personal or work stresses.

A sleep-wake cycle is a repeated cycle of sleep and wake periods. In many normal individuals, the cycle is about 24 hours in duration and has two phases, one characterized by continuous or near continuous sleep, and the other characterized by continuous or near continuous wakefulness.

“Concurrently” as used herein means sufficiently close in time to produce a combined effect (that is, concurrently may be simultaneously, or it may be two or more events occurring within a short time period before or after each other).

“Pharmaceutically acceptable” as used herein means that the compound or composition is suitable for administration to a subject to achieve the treatments described herein, without unduly deleterious side effects in light of the severity of the disease and necessity of the treatment.

“Pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of compounds of the invention.

### B. Active Compounds.

As used herein, “active compounds” are those compounds which relieve one or more of the symptoms of sleep deprivation. Examples include, but are not limited to, a hypocretin agonist (e.g., orexin-A). Other examples of active compounds include, but are not limited to, a potentiator of AMPA receptors such as CX717, caffeine (e.g., NoDoz®, Novartis, Cambridge, Mass.), nicotine, amphetamines, Modafinil (Cephalon, Inc., Frazer, Pa.), etc.

“AMPA receptor potentiators” or “AMPA receptor modulators” are pharmacologic agents that act on the AMPA subtype of glutamate receptors located on neurons and glial cells in the brain and CNS of a subject or patient. Positive AMPA receptor potentiators alter the functional properties of the AMPA receptor, consequently enhancing glutamatergic neurotranmission between neurons and thus facilitating cognitive function when this occurs in critically relevant brain regions. AMPA receptor modulator have been shown to increase neural activity and improve cognitive performance in animal tasks that require both short-term retention and working memory. AMPA receptor potentiators include, but are not limited to, CX717, prolactin releasing peptide agonists, etc. See, e.g., U.S. Patent Application Publication No. 2006/0276462 to Deadwyler ; U.S. Pat. No. 6,884,596 to Civelli

Caffeine is also known, and is found in many products, e.g., NoDoz®, as is nicotine, which is most commonly found in tobacco products.

Amphetamines include, but are not limited to, dextroamphetamine, benzedrine, methylphenidate (Ritalin), etc. Examples of amphetamines include d-amphetamines and l-amphetamines. D-amphetamines primarily potentiate the effects of dopamine in the brain by, e.g., causing the release of dopamine from axon terminals, inhibiting the reuptake of dopamine, etc. L-amphetamines are thought to act primarily on the norepinephrine system.

Modafinil, C₁₅H₁₅NO₂S, is an acetamide derivative thought to modulate the central postsynaptic alpha1-adrenergic receptor, without participation of the dopaminergic system. It has been used to treat, among other things, various sleep disorders involving pathological somnia. See, e.g., U.S. Reissued Pat. No. RE37,516, which is incorporated by reference herein.

Hypocretin is a neuropeptide originally associated with feeding. This neuropeptide is synthesized in neurons of the periformical, dorsomedial, lateral, and posterior hypothalamus (Kiyashchenko, J. Neurophysiol. 85(5):2008-2016 (2001), and commonly exists in one of two different forms: hypocretin-1 (Hcrt-1) and hypocretin-2 (Hcrt-2). The hypocretins are also referred to as orexins (orexin-1 or orexin-A; and orexin-2 or orexin-B). The hypocretin neuropeptides are derived from prepro-hypocretin prepro-orexin), a precursor molecule. Some authors report that prepro-hypocretin and prepro-orexin are the same peptide, but that hypocretins are different from orexins. However, this is a minority view. (Smart, Br. J. Pharmacol. 129(7):1289-91 (2000)). Hypocretin-2 has been reported as a less stable form of hypocretin than hypocretin-1. More stable forms of hypocretin can be made, however, using known techniques. Human orexin-A is reported as a 33 residue peptide of 3562 Da, and human orexin-B is reported as a 28 residue peptide of 2937 Da, and the human prepro-orexin gene has been located at human chromosome 17q21 (Sakurai, Cell 92:573-585 (1998)). The whole length of the human prepro-orexin gene and corresponding cDNA has been cloned by Sakurai, J. Biol. Chem. 274(25):1771-1776 (1999). The human prepro-orexin gene consists of two exons and one intron distributed over 1432 base pairs, and is thought to encode a precursor peptide that is proteolytically processed into two orexin-A and orexin-B.

Reference to hypocretin or orexin (or the precursor) includes the amino acid sequences discussed below, and allelic, cognate, and induced variants thereof. Usually such variants show at least 90% sequence identity to these exemplary sequences. Cognate forms of the human orexin sequence have been cloned from porcine tissues by Dyer, Domest. Anim. Endocrinol. 16(3):1450148 (1999). The term hypocretin also includes fragments of hypocretin peptide having the same or similar functional effect as hypocretin. As used in this application, the term “hypocretin” or “orexin” can also refer to an agonist thereof unless otherwise apparent from the context. Agonists of hypocretin are sometimes referred to as hypocretin analogs. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over the comparison window). Percentage of sequence identity is calculated by comparing two optimally aligned sequences over a window of comparison (typically the entire length of one or both sequences being compared), determining the number of positions at which the identical residues occurs in both sequences to yield the number of matched positions, dividing the number of matched positions by the total number of positions in the window of comparison (i.e., the window size), and multiplying the result by 100 to yield the percentage of sequence identity.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An “agonist” also sometimes known as an analog of a native polypeptide is a compound having a qualitative biological activity in common with the native polypeptide (described in detail below and in US Patent Application No. 2006/0241029 to Siegel). For the purpose of the present invention, an “agonist” of a native Hcrt-1 or Hcrt-2 is defined by their ability to bind to the Hcrt-1 or Hcrt-2 receptor or related polypeptide respectively. For example, an agonist of Hcrt-1 or Hcrt-2 can bind to a native Hcrt-1 or Hcrt-2 receptor or related polypeptide, triggering intracellular events that either cause changes in membrane polarization, cause the release of other neurotransmitters or cause changes in the response to other neurotransmitters. The Hcrt-1 or Hcrt-2 agonists preferably have at least about 60%, more preferably at least about 70%, even more preferably at least about 80%, most preferably at least about 90% overall amino acid sequence identity with a native sequence Hcrt-1 or Hcrt-2 polypeptide, preferably a human Hcrt-1 or Hcrt-2 as described by Sakurai T., 1998, Cell 92:573 85 and de Lecea, L., 1998, Proc. Natl. Acad. Sci. U.S.A. 95:322 327. A sequence of unprocessed human precursor hypocretin protein is provide below from Sakurai, J. Biol. Chem. 274(25):1771-1776 (1999). Orexin A and orexin B occupy residues 34-66 and 70-97 of the precursor

MNLPSTKVSW AAVTLLLLLL LLPPALLSSG AAAQPLPDCC
RQKTCSCRLY ELLHGAGNHA AGILTLGKRR SGPPGLQGRL
QRLLQASGNH AAGILTMGRR AGAEPAPRPC LGRRCSAPAA
ASVAPGGQSG I

Unless otherwise apparent from the context hypocretin refers generically to hypocretin 1 and/or 2 and hypocretin I and orexin A are used interchangeably as are hypocretin 2 and orexin B.

Genbank REFSEQ Accession Nos. NM 001524, NM 001525 and NM 001526 provide cDNA sequences for human hypocretin precursor protein, human hypocretin receptor 1 and human hypocretin receptor 2. Orexin-A (C₁₅₂H₂₄₃N₄₇O₄₄S₄), m.wt. 3561.16 is commercially available from Bachem Biochemical GmbH as cat number H-4172. The Hcrt-1 and Hcrt-2 agonists show at least about 80%, more preferably at least about 90% and most preferably at least about 95% or more amino acid sequence identity with the binding domain of the Hcrt-1 or Hcrt-2 polypeptide sequence, respectively. Fragments of native sequence Hcrt-1 or Hcrt-2 polypeptides from various mammalian species and sequences homologous to such fragments constitute another preferred group of Hcrt-1 and Hcrt-2 agonists. Such fragments preferably show at least about 80%, more preferably at least about 90%, most preferably at least about 95% or more sequence identity with the Hcrt-1 or Hcrt-2 polypeptide sequence. Another preferred group of Hcrt-1 or Hcrt-2 agonists is encoded by nucleic acid hybridizing under stringent conditions to the complement of nucleic acid encoding a native Hcrt-1 or Hcrt-2 polypeptide.

The Hcrt-1 and Hcrt-2 polypeptides of the present invention can be modified to provide a variety of desired attributes, e.g., with improved pharmacological characteristics, while increasing or at least retaining substantially all of the biological activity of the unmodified peptide. For example, the Hcrt-1 and Hcrt-2 peptides or fragments thereof can be modified by extending or decreasing the amino acid sequence of the peptide. Optionally, no more than five amino acids are added or deleted at each end of such a peptide. Substitutions with different amino acids or amino acid mimetics can also be made.

The Hcrt-1 peptides employed in the subject invention need not be identical to peptides disclosed in the Example section, below, so long as the subject peptides are able to induce a same or similar response against the desired Hcrt receptor molecule or related molecule. Thus, a number of conservative substitutions (described in more detail below) can be made without substantially affecting the activity of Hcrt-1 or Hcrt-2.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Single amino acid substitutions, deletions, or insertions can be used to determine which residues are relatively insensitive to modification. Substitutions are preferably made with small, relatively neutral moieties such as Ala, Gly, Pro, or similar residues. The effect of single amino acid substitutions can also be probed using D-amino acids. The number and types of residues which are substituted or added depend on the spacing necessary between essential contact points and certain functional attributes which are sought (e.g., hydrophobicity versus hydrophilicity). Increased activity can also be achieved by such substitutions, compared to the native Hcrt peptide. In any event, such substitutions should employ amino acid residues or other molecular fragments chosen to avoid, for example, steric and charge interference which might disrupt binding.

The substituting amino acids, however, need not be limited to those naturally occurring in proteins, such as L-.alpha.-amino acids, or their D-isomers. The peptides can be substituted with a variety of moieties such as amino acid mimetics well known to those of skill in the art.

The individual residues of the Hcrt polypeptides can be incorporated in the peptide by a peptide bond or peptide bond mimetic. A peptide bond mimetic of the invention includes peptide backbone modifications well known to those skilled in the art. Such modifications include modifications of the amide nitrogen, the .alpha.-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983). See also U.S. Patent Application Publication 2006/0241029 to Siegel, which is incorporated by reference herein.

Amino acid mimetics can also be incorporated in the peptides. An amino acid mimetic as used here is a moiety other than a naturally occurring amino acid that conformationally and functionally serves as a substitute for an amino acid in a polypeptide of the present invention. Such a moiety serves as a substitute for an amino acid residue if it does not interfere with the ability of the peptide to illicit a response against the appropriate Hcrt receptor molecule. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243 252.

As noted above, the peptides employed in the subject invention need not be identical, but can be substantially identical, to the corresponding sequence of the target Hcrt receptor molecule or related molecule. The peptides can be subject to various changes, such as insertions, deletions, and substitutions, either conservative or non-conservative, where such changes might provide for certain advantages in their use. The polypeptides of the invention can be modified in a number of ways so long as they comprise a sequence substantially identical to a sequence in the naturally occurring Hcrt peptide molecule, as described in US Patent Application No. 2006/0241029 to Siegel.

Test compounds can be screened for agonist activity. Compounds constituting agents, conjugates or conjugate moieties to be screened can be naturally occurring or synthetic molecules. Natural sources include sources such as, e.g., marine microorganisms, algae, plants, animals, and fungi. Alternatively, compounds to be screened can be from combinatorial libraries of agents, including peptides or small molecules, or from existing repertories of chemical compounds synthesized in industry, e.g., by the chemical, pharmaceutical, environmental, agricultural, marine, cosmoceutical, drug, and biotechnological industries. Compounds can include, e.g., pharmaceuticals, therapeutics, environmental, agricultural, or industrial agents, pollutants, cosmoceuticals, drugs, organic compounds, lipids, glucocorticoids, antibiotics, peptides, sugars, carbohydrates, and chimeric molecules.

A variety of methods are available for producing peptide libraries (see, e.g., Lam, Nature, 354: 82, 1991 and WO 92/00091; Geysen, J Immunol Meth, 102: 259, 1987: Houghten, Nature, 354: 84, 1991 and WO 92/09300 and Lebl, Int J Pept Prot Res, 41, 201, 1993). Peptide libraries can also be generated by phage display methods. See, e.g., Dower, U.S. Pat. No. 5,723,286.

Combinatorial libraries can be produced for many types of compounds that can be synthesized in a step-by-step fashion (see e.g., Ellman & Bunin, J Amer Chem Soc, 114:10997, 1992 (benzodiazepine template), WO 95/32184 (oxazolone and aminidine template), WO 95/30642 (dihydrobenzopyran template) and WO 95/35278 (pyrrolidine template). Libraries of compounds are usually synthesized by solid phase chemistry on particle. However, solution-phase library synthesis can also be useful. Strategies for combinatorial synthesis are described by Dolle & Nelson, J. Combinatorial Chemistry 1. 235-282 (1999)) (incorporated by reference in its entirety for all purposes). Synthesis is typically performed in a cyclic fashion with a different monomer or other component being added in each round of synthesis. Some methods are performed by successively fractionating an initial pool. For example, a first round of synthesis is performed on all supports. The supports are then divided into two pools and separate synthesis reactions are performed on each pool. The two pools are then further divided, each into a further two pools and so forth. Other methods employ both splitting and repooling. For example, after an initial round of synthesis, a pool of compounds is split into two for separate syntheses in a second round. Thereafter, aliquots from the separate pools are recombined for a third round of synthesis. Split and pool methods result in a pool of mixed compounds. These methods are particularly amenable for tagging as described in more detail below. The size of libraries generated by such methods can vary from 2 different compounds to 104, 106, 108, or 1010, or any range therebetween.

Preparation of encoded libraries is described in a variety of publications including Needels, Proc. Natl. Acad. Sci. USA 1993, 90, 10700; Ni, J. Med. Chem. 1996, 39, 1601, WO 95/12608, WO 93/06121, WO 94/08051, WO 95/35503 and WO 95/30642 (each of which is incorporated by reference in its entirety for all purposes). Methods for synthesizing encoded libraries typically involve a random combinatorial approach and the chemical and/or enzymatic assembly of monomer units. For example, the method typically includes steps of: (a) apportioning a plurality of solid supports among a plurality of reaction vessels; (b) coupling to the supports in each reaction vessel a first monomer and a first tag using different first monomer and tag combinations in each different reaction vessel; (c) pooling the supports; (d) apportioning the supports among a plurality of reaction vessels; (e) coupling to the first monomer a second monomer and coupling to either the solid support or to the first tag a second tag using different second monomer and second tag combinations in each different reaction vessel; and optionally repeating the coupling and apportioning steps with different tags and different monomers one to twenty or more times. The monomer set can be expanded or contracted from step to step; or the monomer set could be changed completely for the next step (e.g., amino acids in one step, nucleosides in another step, carbohydrates in another step). A monomer unit for peptide synthesis, for example, can include single amino acids or larger peptide units, or both.

Compounds synthesizable by such methods include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Prepared combinatorial libraries are also available from commercial sources (e.g., ChemRx, South San Francisco, Calif.).

There are known methods for detecting, identifying, or screening for hypocretin agonists or antagonists. Typically, such methods rely on the observation that activation of hypocretin receptor is associated with an increase in intracellular calcium ([Ca2+]) concentrations (Smart, Br. J. Pharmacol. 129(7):1289-91 (2000); Kukkonen, Neuroreport 12(9):2017-20 (2001); and Lund, J. Biol. Chem. 275(40):30806-12 (2000)).

One procedure involves cell lines such as Chinese hamster ovary-K1 cells, and includes a first set of cells that are transfected with hypocretin receptors, and a second set of cells that are not transfected with hypocretin receptors. Each of the first and second sets of cells are evaluated to determine their response to hypocretin or a known agonist thereof. Further, each of the first and second sets of cells are evaluated to determine their response to a test compound. It is assumed that in response to hypocretin or a known agonist thereof, the transfected cells will show a greater response than the nontransfected cells. The test compound is evaluated as to whether it shows a activity profile similar to that of hypocretin or the known agonist. Table 1 below illustrates possible outcomes of this procedure.

TABLE 1

| | | | hypocretin or |
|---|---|---|---|
| | | test compound | known agonist |
| Neg. | nontransfected cell | no response | no response |
| | transfected cell | no response | response |
| Pos. | nontransfected cell | no response | no response |
| | transfected cell | response | response |

In the first trial, the hypocretin or known agonist thereof shows a response in the transfected cell, but not in the nontransfected cell, as expected. Because the test compound shows no response in either of the sets of cells, the result is negative: the test compound is not a hypocretin agonist. In the second trial, because the test compound shows an activity profile similar to the hypocretin or known agonist thereof, the result is positive: the test compound is a hypocretin agonist.

Many of these assay procedures use calcium imaging, although other methods are available. For example, assays that measure changes in the cell membrane potential can be used. Related approaches include current clamp and voltage clamp techniques. These assays can be performed in association with manipulation of a bathing medium to determine which ionic currents are affected by a substance. Likewise, a wide variety of other techniques exist for assessing the biological effects of test molecules in relation to those of hypocretin or known agonists thereof Further, in addition to Chinese hamster ovary-K1 cells, other cell lines can be used, such as neurons cultured from rat medial and lateral hypothalamus (van den Pol, J. Neurosci. 18(19):7962-71 (1998)) and spinal cord (van den Pol, J. Neurosci. 19(8):3171-82 (1999)).

Hypocretin agonists and antagonists may also be identified using structural analysis. Recent studies indicate that certain regions of the hypocretin polypeptide are important for ligand-receptor interaction (Darker, Bioorg. Med. Chem. Lett. 11(5):737-40 (2001)). Relatedly, computer aided drug design can be used in the evaluation or development of test compounds with regard to their hypocretin agonist activity. Computer programs such as Dock, Frodo, and Insight can be used to aid in the design and development of peptides, peptidomimetics, and small molecules that interact with the hypocretin receptor.

The term specific binding (and equivalent phrases) refers to the ability of a binding moiety (e.g., a receptor, antibody, Hcrt-1 or Hcrt-2 agonist, ligand or antiligand) to bind preferentially to a particular target molecule (e.g., ligand or antigen) in the presence of a heterogeneous population of proteins and other biologics (i.e., without significant binding to other components present in a test sample). Typically, specific binding between two entities, such as a ligand and a receptor, means a binding affinity of at least about 10⁶ M⁻¹, and preferably at least about 10⁷, 10⁸, 10⁹, or 10¹⁰ M⁻¹. Specific (or selective) binding can be assayed (and specific binding molecules identified) according to the method of U.S. Pat. No. 5,622,699; this reference and all references cited therein are incorporated herein by reference. Typically a specific or selective reaction according to this assay is at least about twice background signal or noise and more typically at least about 5 or at least about 100 times background, or more.

As noted above, active compounds or active agents of the present invention include prodrugs of the foregoing. Such prodrugs are, in general, compounds that are rapidly transformed in vivo to yield a polypeptide as described above, for example, by hydrolysis in blood. A thorough discussion is provided in T. Higuchi and V. Stella, Prodrugs as Novel Delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

The active compounds disclosed herein (including prodrugs) can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

### C. Pharmaceutical Formulations.

The active compounds described herein can be formulated for administration in a pharmaceutically or physiologically acceptable carrier, diluent or excipient in accordance with known techniques. See, e.g., Remington, The Science And Practice of Pharmacy (9th Ed. 1995). In the manufacture of active compounds according to the invention, the compounds (e.g., peptides) are admixed with, inter alia, an acceptable carrier. The carrier must, of course, be acceptable in the sense of being compatible with any other ingredients in the formulation and must not be deleterious to the patient. The carrier can be a solid or a liquid, or both, and is preferably formulated with the compound as a unit-dose formulation, for example, a tablet, which may contain from 0.01 or 0.5% to 95% or 99% by weight of the active compound. One or more compounds of interest and/or targeting compounds can be incorporated in the formulations of the invention.

The formulations of the invention include those suitable for oral, rectal, topical (i.e., skin, hair, nails, hooves, both skin and mucosal surfaces, including airway surfaces), buccal (e.g., sub-lingual), vaginal, parenteral, transdermal, nasal, and inhalational administration, although the most suitable route in any given case will depend on the nature and severity of the condition being treated and on the nature of the particular cells of interest being targeted.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound, or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active agents or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

### D. Dosage and Routes of Administration.

Active compounds as described herein may be administered (typically as a pharmaceutical formulation as described herein) by any suitable route of delivery, including but not limited to oral administration, topical application, transdermal administration, buccal administration, rectal administration, or parenteral administration (e.g., intramuscular, intradermal, intravenous, intraperitoneal, or subcutaneous injection).

The therapeutically effective dosage of any specific compound, the use of which is in the scope of the present invention, will vary somewhat from compound to compound, and patient to patient, and will depend upon the condition of the patient and the route of delivery. As a general proposition, a dosage from about 0.01, 0.1, 0.5, 1, 5 or 10 micrograms per kilogram, up to 1, 10 or even 50 mg/kg will have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active compound, including the cases where a salt is employed. A dosage from about 10 mg/kg to about 50 mg/kg may be employed for oral administration. A dosage from about 0.5 mg/kg to 5 mg/kg may be employed for intramuscular injection. Exemplary dosages for nasal administration include 0.01-100, 0.1 to 10, 0.1-5, 0.5-5, 0.5-2, or 1 μg orexin A or molar equivalent of other agonist, optionally with normalization to accommodate any differences in potency between the agonist and orexin-A. Exemplary dosages for nasal administration also include 0.1-10, 0.1-5 or 1 μg/kg. As a practical matter, suitable dosages can be administered by one or two squirts of mist with orexin-A at a concentration of 1-100, 10-50 or 25 μg/ml. The duration of the treatment can be one time or more per day for a period of two to three weeks or until the condition is essentially controlled. Lower doses given less frequently can be used prophylactically to prevent or reduce the incidence of recurrence of the sleepiness.

### E. Exemplary Regimes

The data provided in the Examples show that administration of hypocretin can result in a rapid (e.g., within five minutes) improvement in cognitive function in sleep-derived patients, and that the improvement is particularly marked in performing tasks with a high cognitive load. Accordingly, the timing of administration of hypocretin or an agonist can be customized in a patient depending on when the patient needs to perform tasks whose performance would otherwise be impaired by sleep deprivation. For example, hyocretin can be administered within 5 min, 10 min, 30 min or an hour of beginning such a task or during performance of a task. If the effects of a first dose wear off while performing a task, one or more further doses can be administered until the task is completed.

Such a pattern of administration responsive to the need to perform particular tasks can result in administration of hypocretin or an analog at irregular intervals to a subject. For example, administration can be confined to particular days on which the subject has to undertake a task of relatively high cognitive load.

Cognitive load is the level of perceived mental effort associated with thinking and reasoning in an individual. Cognitive load can depend on both the individual and the task. Means of measuring cognitive load include: subjective measures, such as self-rating scales; physiological techniques, such as pupil dilatation and heart rate; task or performance based measures, such as critical error rates; and behavioral measures, such as speech disfluencies and self-talk. A task with a high cognitive load means a task requiring above average mental function for an individual during that individuals waking existence, such as performing a skilled or professional occupation, making a decision based on skill, memory or professional judgment, performing a task requiring hand-eye coordination, or remembering or recalling sequences or categories of stimuli.

The compositions and methods are useful for individuals not diagnosed with a particular sleep disorder or having any know biochemical or genetic marker indicating susceptibility to such a disorder. Thus, in some methods, the subject is free of narcolepsy, REM sleep behavior disorder, period leg movements in sleep and restless leg syndrome, circadian rhythm disorder, sleep apnea, hypersomnia, insomnia, Alzheimer's disease, depression, schizophrenia and obesity. Such individuals do not generally have difficulty in maintaining consolidation of appropriate sleeping and waking periods, but can nevertheless suffer occasional sleep deprivation due to real or imagined stresses of work or personal lives. Administration of hypocretin or an analog in such individuals at irregular intervals when sleep deprivation occurs can be useful.

The regime used to treat symptoms of sleep deprivation can differ from that used to treat an underlying sleep disorder. In the latter case, the goal is often to promote a regular cycle with continuous consolidated sleep and wake periods. Such a goal is promoted by administration of hypocretin at regular intervals (e.g., once per day, once every other day or once per week). By contrast, a goal in treating symptoms of sleep-deprivation is to address an immediate problem, which is often present in individuals both with and without any underlying sleep disorder. Such a goal can accomplished by administration of hypocretin at irregular intervals and/or dosage depending on such factors as tasks requiring high cognitive function or transient stresses or increased work loads contrasts. Such a pattern of administration may serve to extend the wake period beyond normal range and introduce a temporary alteration in a sleep-wake cycle in person with an otherwise normal circadian rhythm.

### F. Nasal Administration and Devices.

In preferred embodiments, active compounds such as orexin-A and its agonists are administered via the nasal route, in a calculable dose that is effective via this route of administration. As described below, this compound delivered in this manner has been shown to reverse the effects of sleep deprivation. When compared with intravenous delivery of orexin-A (Tocris Bioscience in 500 μg (powder), catalog #1455 orexin-A, Human Rat, Mouse synthesized from the peptide sequence) under similar testing conditions, an atomizer spray containing the compound was more effective. The atomizer may be, e.g., a commercially available, glass-based atomizer (Devilbiss Model #163, available from Micromedics, Inc., St. Paul, Minn.). In some embodiments the glass vial base is pretreated to prevent peptide sticking. Without wishing to be bound to any particular theory, it is thought that the peptide does not effectively cross the blood brain barrier and cannot be delivered in blood because of dosage inaccuracies.

In some embodiments, instead of a pressurized atomizer, the invention utilizes a small container (e.g., plastic container) that can be squeezed to expel a mist spray into the nostrils of a subject (e.g., human subjects). Appropriate concentrations and volumes of delivery may be determined by routine procedures by one skilled in the art by, e.g., determining permeability of the nasal mucosa with nasal or oral inhalation, and may be calculated based upon, e.g., studies in non-human primates.

Active agents according to the present invention may further include a pharmaceutically acceptable carrier suitable for nasal administration. Such carriers include, but are not limited to, solid (e.g., particulate) and aqueous carriers. See, e.g., U.S. Pat. No. 4,613,500 to Suzuki; U.S. Pat. No. 5,629,011 to Illum; U.S. Pat. No. RE36,744 to Goldberg; and U.S. Pat. No. 5,628,984 to Boucher, Jr. In some embodiments, a particulate size of between 10 and 500 μm is preferred to ensure retention in the nasal cavity.

Solid particulate compositions containing dry particles of micronized active compounds may be prepared by, e.g., grinding dry active compound with a mortar and pestle, and then passing the micronized composition through a 400 mesh screen to break up or separate out large agglomerates. A solid particulate composition comprised of the active compounds may optionally contain a dispersant which serves to facilitate the formation of an aerosol. A suitable dispersant is lactose, which may be blended with the active compound in any suitable ratio (e.g., a 1 to 1 ratio by weight).

Aerosols of liquid particles including the active compound may be produced by any suitable means, such as with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or a dilute aqueous alcoholic solution, preferably made isotonic with body fluids by the addition of, for example, sodium chloride. Optional additives include preservatives if the formulation is not made sterile, for example, methyl hydroxybenzoate, antioxidants, flavoring agents, volatile oils, buffering agents and surfactants.

Aerosols of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a sniff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active compound or of a powder blend including the active compound, a suitable powder diluent, such as lactose, and an optional surfactant. The active compound in some embodiments comprises from 0.1 to 100 w/w of the formulation. A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 150 μl, to produce a fine particle spray containing the active compound. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

### G. Combination Treatments and Compositions.

In another embodiment, an active compound of the invention is used in combination with other therapeutic modalities, for example in like manner as described in U.S. Pat. No. 6,946,441 to Long Thus, in addition to the therapies described above, one may also provide to the patient more “standard” pharmaceutical anti-inflammatory therapies. Examples of standard therapies include, without limitation the concurrent administration of an additional active compound, separately or in the same pharmaceutical formulation as containing the active compounds described herein.

In some embodiments, the active compound of the invention may precede or follow administration of the other agent by intervals ranging from minutes to weeks. In embodiments where the compounds are applied separately to the cell, one would generally ensure that a significant period of time did not expire between the time of each delivery, such that each active compound would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In some embodiments, more than one administration of either an active compound of the invention, or the other agent will be desired. In this regard, various combinations may be employed. By way of illustration, where the active compound is “A” and the other agent is “B”, the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated.

### H. Treatment of Parkinson's Disease

Parkinson's disease is one of a family of related diseases termed Lewy Body diseases (LBD). These diseases are characterized by degeneration of the dopaminergic system, motor alterations, cognitive impairment, and formation of Lewy bodies (LBs). (McKeith, Clinical and pathological diagnosis of dementia with Lewy bodies (DLB): Report of the CDLB International Workshop, Neurology (1996) 47:1113-24). Other LBDs include diffuse Lewy body disease (DLBD), Lewy body variant of Alzheimer's disease (LBVAD), combined PD and Alzheimer's disease (AD), and multiple systems atrophy.

The hypocretin and analogs, and formulations thereof described in connection with treating symptoms of sleep deprivation can also be used mutatis mutandis in treatment of Parkinson's disease or other Lewy body disease and one or more symptoms of such diseases. As discussed in Example 2, Parkinson's disease patients have reduced levels of hypocretin and narcolepsy-like symptoms. Administration of hypocretin or an analog can be used to treat any or all of the following symptoms in such patients: daytime sleep attacks, nocturnal insomnia, a REM sleep behavior disorder, a hallucination or depression, and combinations thereof Hypocretin or an analog is administered in a regime effective to ameliorate at least partially at least one symptom of Parkinson's disease. Treatment of Parkinson's patients can be performed using regimes (i.e., dosage, frequency and route of administration) as those described above or previously described in conjunction with narcolepsy, (see U.S. Pat. No. 7,112,566). For example, treatment can be administered on a daily basis, every two days or weekly. A typical dose range is 0.01 to 20 or 0.1-5 μg/kg. Absolute dosages ranging for example from 0.1-500, 0.1-10, 0.5-5 0.5-5, 0.5-2 or 1 μg are also suitable in treating Parkinson's as with other methods of the invention described above. Dosages are given based on administration of orexin-A. Other agonists can be administered on an equivalent molar ratio with normalization for any difference in efficacy between the agonist and orexin A. Hypocretin can be administered peripherally or directly into the CNS. Nasal delivery, optionally, via a spray, within dosage ranges indicated above is particularly preferred, as described above. Nasal delivery of hyocretin in human subjects for treating olfactory dysfunction has been reported (Baier, Brain (August 2008)).

Hypocretin or an analog can optionally be administered with another compound that acts on another biochemical deficit that may be present in a Parkinson's disease patient. Examples of such compounds include dopamine agonists (Parlodel (bromocriptine), Dostinex (cabergoline), Permax (pergolide), Mirapex (pramipexole), Requip (ropinirole), Apokyn (apomorphine), Neupro (rotigotine), serotonin selective reuptake inhibitors (e.g., citalopram, dapoxetine, escitalopram, fluoxetine, fluvoxamine, paroxetine, sertraline, zimelidine) and norepinephrine reuptake inhibitors (e.g., Atomoxetine (Strattera), Reboxetine (Edronax), Viloxazine (Vivalan), Maprotiline (Ludiomil), Nortriptyline (Nortrilen), Bupropion (Wellbutrin) and Radafaxine.

### I. Diagnosing and Monitoring Parkinson's Disease

The presence and clinical stage of Parkinson's disease can be assessed by measuring a reduction in hypocretin levels in a patient. Hypocretin in plasma or the CSF can be measured by radioimmunoassay (RIA), as described by Nishino, Lancet 355:39-40 (2000). The radioimmunoassays involve competition of ¹²⁵I-hypocretin and standard or test samples for limited quantities of hypocretin-specific antibody. If the standard or test sample contains a lower amount of hypocretin, then a higher amount of ¹²⁵I-hypocretin can bind to the antibody. Conversely, if the standard or test sample contains a higher amount of hypocretin, then a lower amount of ¹²⁵I-hypocretin can bind to the antibody. Standard curves can be constructed by measuring the amount of bound ¹²⁵I-hypocretin as a function of hypocretin in a standard reaction. The concentration of hypocretin in a test sample can then be determined using the standard curve. Assay kits such as these are commercially available (e.g. Phoenix Pharmaceuticals, 530 Harbor Blvd, Belmont, Calif. USA).

A measured level of hypocretin in a patient is compared with a level determined previously in the patient, with levels determined in a series of patients with different stages of Parkinson's disease or with a control population of patients unaffected by Parkinson's disease. A reduction relative to a previous measured level in the same patient indicates a more advanced stage of disease. Comparison with a series of measurements in patents with different stages of Parkinson's disease allows interpolation of the stage of disease in the patient. The existence of and magnitude of a reduction compared with an average level in control patients not having Parkinson's disease provide an indication of presence and clinical stage of Parkinson's disease.

The present invention is explained in greater detail in the following non-limiting Examples.

### Experimental

### Example 1

### Treatment of Sleep Deprivation and Cognitive Impairment

The effects of administering orexin-A to monkeys engaged in a cognitive task following 30-36 hrs of sleep deprivation are described, and the resulting changes in rates of local cerebral glucose metabolism (CMRglc) noted. This is the first instance in which Orexin-A has been employed via a novel application method to provoke increased levels of this peptide in the brain of nonhuman primates. We have determined that orexin-A applied via intranasal delivery can counteract the effects of sleep deprivation in nonhuman primates and that this procedure will have positive implications for adapting it to use in humans.

Hypocretin-1 (orexin-A) was administered to sleep deprived (30-36 hrs) rhesus monkeys immediately preceding testing on a multi-image delayed match to sample (DMS) short-term memory task. The DMS task employed multiple delays and numbers of stimulus images and has been shown to effectively measure cognitive defects produced by sleep deprivation (Porrino 2005). Two methods of administration of orexin-A were tested, intravenous injections with a dose range 2.5-10.0 μg/kg of and a novel method developed for nasal delivery via an atomizer spray mist (dose estimated 1.0 μg) to the nostrils. Results showed that orexin-A delivered via both the IV and nasal routes significantly improved performance, however, the nasal delivery method was significantly more effective than the highest IV dose (10 μg/kg) tested in reversing the effects of sleep deprivation. The reversal of sleep deprivation effects on performance by orexin-A was specific to trials classified as high vs. low cognitive load as determined by performance difficulty under normal testing conditions. Surprisingly, neither delivery method of orexin-A affected task performance if animals were tested when alert and not sleep deprived. The behavioral findings were supported by alterations in local cerebral glucose metabolism (CMRglc) in specific brain regions shown to be engaged by the task and impaired by sleep deprivation (Porrino 2005). Analyses indicated that the orexin-A induced recovery of DMS performance resulted from a reversal of sleep deprivation induced changes in local CMRglc in these same brain regions. Consistent with the differential effects on performance, nasal delivered orexin-A produced a more pronounced reversal of sleep induced changes in brain metabolic activity than IV orexin-A.

Subjects: All procedures were reviewed and approved by the Institutional Animal Care and Use Committee of Wake Forest University, and performed in accordance with established practices as described in the National Institutes of Health Guide for Care and Use of Laboratory Animals. Eight adult male rhesus monkeys (Macaca mulatta) weighing (8.0-11.0 kg) were utilized in this study. They were individually housed in stainless steel cages in temperature and humidity controlled colony rooms with lighting maintained on a 0600:1800 on:off schedule and fed a diet of monkey chow supplemented by fresh fruit to maintain daily monitored body weight. Fluid intake was restricted in time and amount such that a prescribed volume of an animal's normal daily fluid intake (80 ml/kg) was received either during the behavioral testing session, or within 2 hours of being returned to the home cage.

Behavioral Testing: Each monkey was exposed to 6 different testing conditions (Normal Vehicle, Alert orexin-A (IV and nasal), Sleep Deprivation, Sleep Deprivation+orexin-A (IV or nasal) which required 11-14 test sessions for each of the 8 monkeys. Animals were placed in a primate chair 1.5 meters in front of an LCD-front-projection screen for daily testing on a multi-image visual delayed match to sample (DMS) task (Porrino 2005) and performed 150-300 trials per session. Animals were trained to move a cursor tracked by a fluorescent marker attached to the back of the monkey's hand into the images by positioning the hand within a two dimensional coordinate system on the chair counter. Stimuli consisted of clip art images projected as 25 cm squares within a 3×3 position matrix onto a 1.0 meter×1.0 meter display. Responses to appropriate stimuli were rewarded with diluted fruit juice delivered via a sipper tube placed in front of the mouth. All animals were trained to a stable baseline on the DMS task in which delay varied randomly from (range 1-60 sec to 1-90 sec across animals) on a given trial, and the number of non-match stimuli (images) varied randomly from 2 to 8 in the Match phase of the task (Porrino 2005). As shown previously performance accuracy varied directly with duration of delay and number of nonmatch images (#images) presented in the Match phase (Hampson 2004, Porrino 2005). Each stimulus image (sample or nonmatch) was used on only one trial during a daily session and the sets of images were routinely changed every two weeks to maintain the trial unique feature of each session. Performance was monitored daily on the DMS task and there was no testing on weekends.

Sleep Deprivation Procedure: Sleep deprivation consisted of 30-36 hours of continuous sleep prevention supervised by laboratory personnel as previously verified using EEG recordings and sleep architecture to be a valid method of depriving animals of slow wave and REM sleep (Porrino 2005). Animals were maintained in a cage separate from their home cage in a continuously lighted room and kept awake with videos, music, occasional treats, gentle cage shaking, and interaction with technicians until their usual daily testing time. Animals were allowed 10 days between sleep deprivation episodes and were allowed to sleep after testing on each day following the 30-36 hr sleep deprivation episode. There were no residual effects of sleep deprivation on testing 24 hrs after the sleep deprivation procedure and animals returned to there normal sleep patterns on the next night following the sleep deprivation procedure.

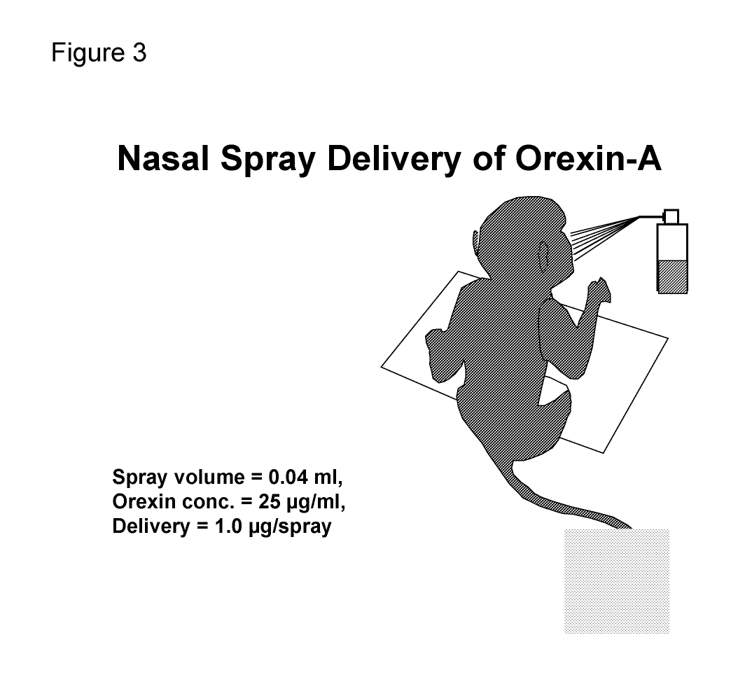

Drug Administration: orexin-A (hypocretin-1) was mixed in saline for IV injections and in sterile water for nasal application. For IV administration orexin-A, (#003-30, Phoenix Pharmaceuticals, Mountain View, Calif.) was dissolved in physiological saline as a stock solution of 200 μg/ml which was then diluted in saline to inject 3 different doses of 2.5, 5.0 and 10.0 μg/kg via an indwelling catheter placed in either the femoral or jugular vein using a glass syringe. The glass syringe was pre-soaked in 1% BSA, rinsed in Milli-Q water then dried at 60° C. prior to use. Only the highest dose of orexin-A (10.0 μg/kg) was administered IV to sleep deprived animals. Nasal orexin-A was administered via an atomizer that was operated via computer controlled pressure valve that controlled delivery to 50 ms epochs via a spray delivered via the tip of the atomizer placed within 2-4 cm of the animals face in the region of the nostrils. Two 50 ms spray bursts were delivered to animal on each occasion of nasal orexin-A administration. The concentration of orexin-A in the spray solution is shown in FIG. 3 along with a picture of the atomizer and the method of delivery just prior to the onset of behavioral testing. orexin-A was mixed to a concentration of 25 μg/ml in sterile water and then loaded in a volume of 30.0 ml into the glass vial base of the atomizer. Animals were habituated to the spray delivery method by subjecting them routinely to similar sprays of saline or sterile water immediately prior to testing on nearly every day during the study. Delivery occurred within 5 min of the start of testing in a cognitive task. The atomizer contained a long (7.0 cm) metal delivery tube (1.0 mm diameter) that could be positioned 4.0-8.0 cm in front of the monkey's nostrils and then pulsed from operation of a switch that activated the computer timed pulse to open the valve and spray the contents in a mist cloud of approximately 4.0-6.0 cm in diameter. The spray mist was estimated to contain approximately 1.0 μg orexin-A in a volume estimated to be 0.04 ml of sterile water. This calculates roughly to a dose of 1.0 μg per spray, depending on how much mist was inhaled. Monkeys typically received one or two squirts of the mist depending on whether they turned their heads as the mist was delivered.

Measurement of Local Rates of Cerebral Glucose Metabolism: Measurement of rates of cerebral glucose metabolism (CMRglc) were made in 8 monkeys in four different conditions: normal alert DMS task vehicle; DMS task-Sleep Deprivation vehicle; DMS task-Sleep Deprivation+orexin A (IV or nasal). All animals (n=8) were acclimated to the PET scan sessions to the extent that performance was not influenced by the injection or blood collection procedures. On the day of the scan the monkeys were placed in primate chairs and received a 30 sec injection of 3-5 mCi of [¹⁸]-2-deoxy-2-fluoro-D-glucose (FDG). The DMS task was initiated 5-10 min later and was performed for a total of 40 minutes (80-100) trials while the FDG was incorporated and taken up by cells during performance in the DMS task. After 40 min animals were then anesthetized with ketamine (15 mg/kg, iv) and transported to the PET scanner. Following scan acquisition, monkeys were transported back to their home cages and continuously monitored until fully recovered. Effects of anesthesia and other factors associated with the PET scanning procedures were previously assessed and shown to not influence measures of isotope uptake.

To calculate glucose uptake tracer concentrations were measured in blood samples using an automated gamma well counter from three blood samples collected from the femoral vein at 10 minutes prior to tracer injection and 8 and 42 minutes post-injection. A population-averaged FDG blood curve (calculated for monkey) was scaled to the measured blood curve for the time period from injection to the end of the PET scan (Sokoloff 1977). Data were transformed to CMRglc based on the operational equation (Phelps 1979, Kennedy 1978). Rate constants (k1, k2, and k3) and lumped constant as determined in monkey by Kennedy et. al. 1978, along with a k4 value used in human studies (Phelps 1979). PET scans were performed with a General Electric (GE) Advance NXi PET scanner with a resolution of 4 mm and consisted of a five minute transmission scan acquired in 2D mode, followed by a ten minute emission scan acquired in 3D mode. The image reconstruction of the 3D data used the 3D-reprojection method with full quantitative corrections and smoothed using a 4 mm Gaussian filter transaxially and then segmented. Data were corrected for attenuation and reconstructed into 128 by 128 matrices using a Hanning filter with a 4mm cutoff transaxially and a ramp filter with an 8.5 mm cutoff axially.

PET data were analyzed with Statistical Parametric Mapping (SPM99) software (http://www.fil.ion.ucl.ac.uk/spm/) implemented in MATLAB (MathWorks, Natick, Mass.). Reconstructed images for each scan from each of the 8 monkeys were co-registered to corresponding structural MR images obtained on a 1.5-T MR scanner (GE Medical Systems, Milwaukee, Wis.) using automated image registration (Black 2001) and then transformed spatially into a standard space with an FDG template for rhesus monkeys constructed in our laboratory based on procedures of Black and colleagues (Paxinos 2003). Resultant images were smoothed using a 2 mm isotropic Gaussian kernel with a voxel size of 1×1×1 mm. Scans were normalized for differences in global activity by proportional scaling. Effects at each voxel were estimated according to the general linear model using the multi-subject conditions and covariates option in SPM. Statistical maps were created for comparisons of the five different experimental conditions: Normal Alert Vehicle vs. Normal Alert orexin-A (IV or nasal); Sleep Deprivation vs. Normal Alert Vehicle; Sleep Deprivation vs. Sleep Deprivation+orexin-A (IV or nasal) and Sleep Deprivation+orexin-A IV vs. Sleep Deprivation+orexin-A nasal. Exploratory analyses used a minimum voxel height (magnitude) threshold of p<0.01 and a minimum cluster size of 50 voxels. A region of interest analysis was conducted including dorsal prefrontal cortex (DPFC), medial temporal lobe (MTL), parietal cortex (precuneus), thalamus, and dorsal striatum, selected based on previous analyses in these same contexts (Porrino 2005). Spherical ROIs were constructed on a structural MR template using the MarsBaR toolbox contained in SPM. Statistical significance for the 4 comparisons described above was determined with a threshold value of p<0.05 corrected for the search volume. Areas of activation were displayed on a T1 weighted anatomic MRI template then identified with reference to standard atlases of the primate brain (Paxinos 2003).

### Effects of Orexin-A Delivered Systemically and Intranasal on Performance Disrupted by Sleep Deprivation

The propensity for orexin-A to alleviate the effects of sleep deprivation was examined by administering the peptide to monkeys that had been sleep deprived 30-36 hrs prior to testing in the DMS paradigm. orexin-A was administered either IV or via nasal spray 10 min prior to testing on the day following a single night of sleep deprivation. Animals were also tested for the effects of orexin-A when performing the task in the alert state after a normal 24 hr sleep cycle. Results are described below in relation to 1) effects of orexin-A on performance in sleep deprived monkeys and 2) comparison of orexin-A delivered either IV or via nasal spray with respect to alleviating the effects of sleep deprivation on DMS performance. In addition effects of orexin-A on local cerebral glucose metabolism (CMRglc) are presented corresponding to each of the above test conditions.

### Performance of Multi-Image DMS Task by Alert Non-Sleep Deprived Monkeys:

Performance of the DMS task has been described in detail in prior publications (Hampson 2004, Porrino 2005) and was not different in the animals tested in this study except that the delay parameters and number of objects were increased over the prior testing conditions because of animal familiarity with the task. As stated in Methods data from each DMS session was classified on the basis of trial rankings with respect to cognitive load: long duration (60-120 sec) trials with more images (6-8) comprised high cognitive load (high load) trials; trials with short delays (1-15 sec) and few images (2-4) constituted low cognitive load (low load) trials. In addition the mean over all trials is also shown for comparison, Normal performance was assessed in animals tested after a standard sleep period with normal light dark cycle with saline vehicle administered either IV or via nasal spray 5-8 min prior to testing (saline controls). All comparisons are made to vehicle (saline) sessions interspersed between orexin-A testing or sleep deprivation sessions within the same weekly testing period. At least one saline control session was interspersed between each orexin-A test session. At least 10 days separated exposures to sleep deprivation procedures in each monkey as per ACUC regulations.

### Effects of Systemic Orexin-A on DMS Performance in Alert Monkeys

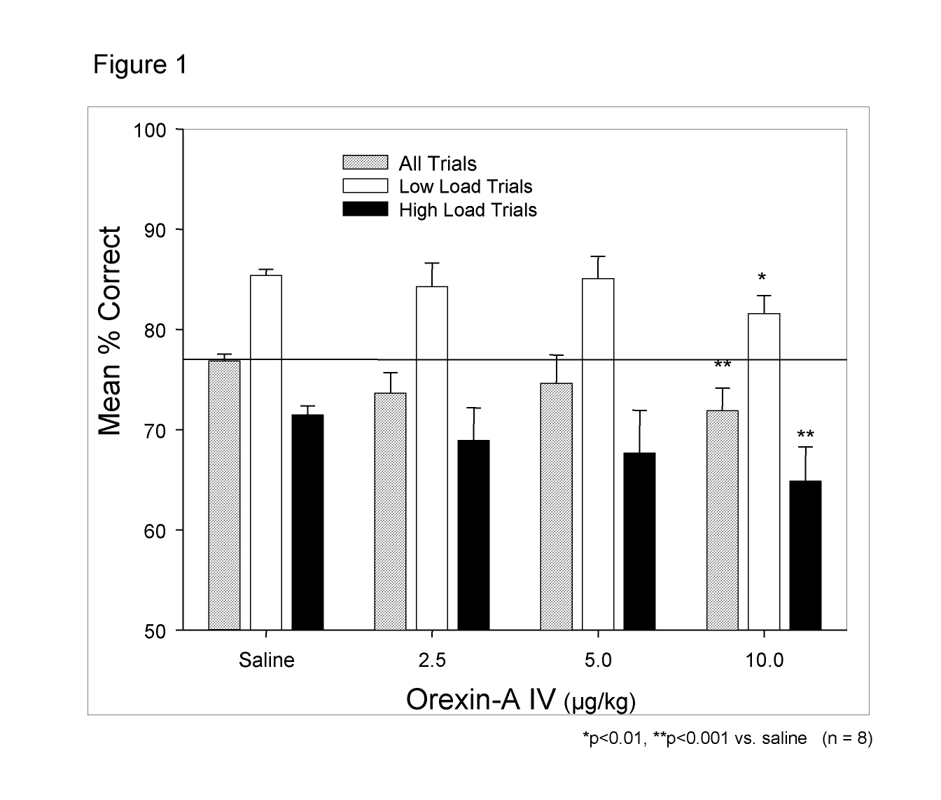

Orexin-A was administered IV in three different doses (2.5, 5.0 and 10.0 μg/kg) to non-sleep-deprived alert animals immediately prior to testing. FIG. 1 shows that there was a clear dose-related trend toward reduced performance as the dose increased from 2.5 μg/kg and but did not reach significance until the at the 10.0 μg/kg level (10 μg/kg dose compared to saline control; however, all 8 monkeys showed a significant reduction at this dose level. Statistical assessment via Analysis of Variance (ANOVA) revealed effects of dose on All trials: F(1,503)=6.81, p<0.001; on Lo-load trials: F(1,503)=5.77, p<0.01; and on Hi-load trials: F(1,503)=11.25, p<0.001).

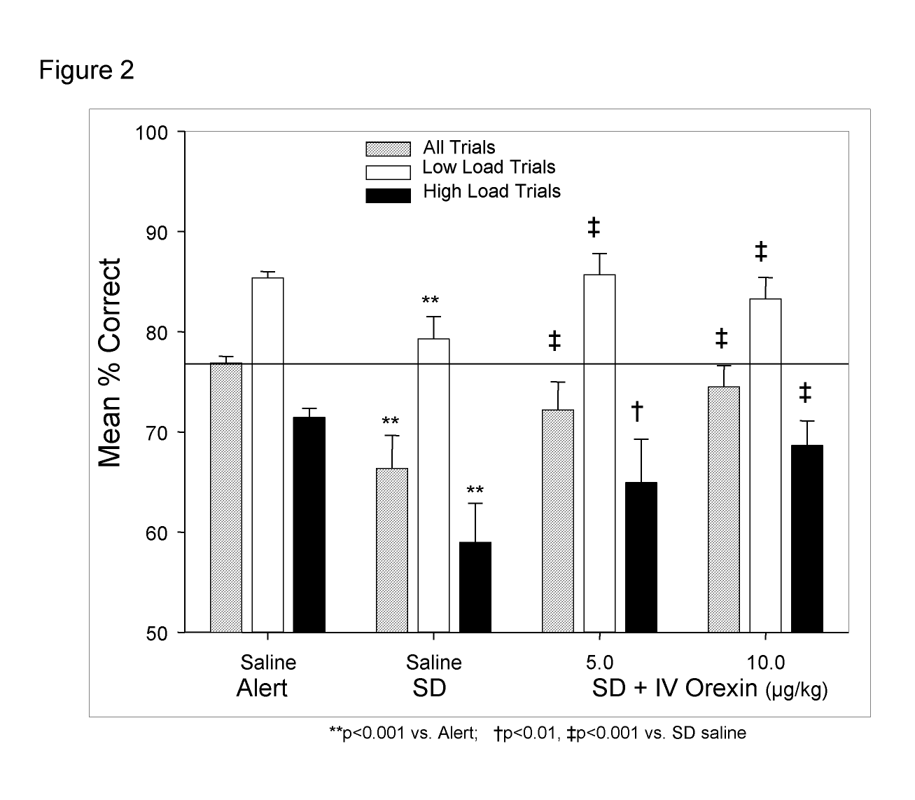

Effects of IV Administered orexin-A on Sleep Deprived Performance: All animals received intravenous injections of orexin-A at the 5.0 and 10.0 μg/kg dose prior to DMS testing after being sleep deprived for 30-36 hrs. FIG. 2 shows that the two doses (5.0 and 10.0 μg/kg) systematically increased performance over sleep deprived alone vehicle (saline) sessions (Overall: F(_{1,503})>15.96, p<0.001; Lo-load trials: F(_{1,503})>12.63, p<0.001; Hi-load trials F(_{1,503})>6.92, p<0.01), however neither dose restored performance to alert non-sleep deprived levels. FIG. 2 also shows a differential effect of the orexin-A on high vs. low cognitive load trials, with low cognitive load trials performed with the same efficiency as in the alert condition (F(_{1,503})=2.51, N.S.) under both doses whereas high load trials showed a residual effect of sleep deprivation even though performance on high load trials was significantly improved relative to sleep deprived sessions (5.0 and 10.0 μg/kg doses high vs. low load trials, F(_{1,503})=7.01, p<0.01). Only one of 8 sleep deprived monkeys failed to show significant improvement in performance on high load trials following the 10.0 μg/kg dose administration orexin-A.

Effects of Nasal Delivered orexin-A on Sleep Deprived Performance: Orexin-A was mixed in a saline solution and sprayed in a mist through a pulsed pressurized atomizer held directly in front of the monkey's nostrils. The pulse duration was controlled by a computer and was set to 50 ms. The concentration of orexin-A in the atomizer vial was 25 μg/ml and the estimated spray volume was 0.04 ml, yielding a delivered dose estimate of 1.0 μg/spray. Two instances of a 50 ms spray delivered 2-5 sec apart and within 5-10 cm of the animals nostrils was administered prior 3-5 min prior to testing in both alert normal as well as following 30-36 hr of sleep deprivation. Each animal was thoroughly habituated to the procedure by administering the spray containing only saline or sterile water in the same manner prior to every control (vehicle) session reported, including those preceding non-drug sleep deprivation sessions. Since each animal received the same exposure to the vehicle spray over nearly all daily testing sessions, the spray containing orexin-A could be delivered in a consistent manner to each subject and exposure by his method was not different for either drug or vehicle. Comparisons of sleep deprived sessions were always with respect to saline or vehicle spray administered to the animal in the same manner as in the orexin-A sleep deprivation test sessions.

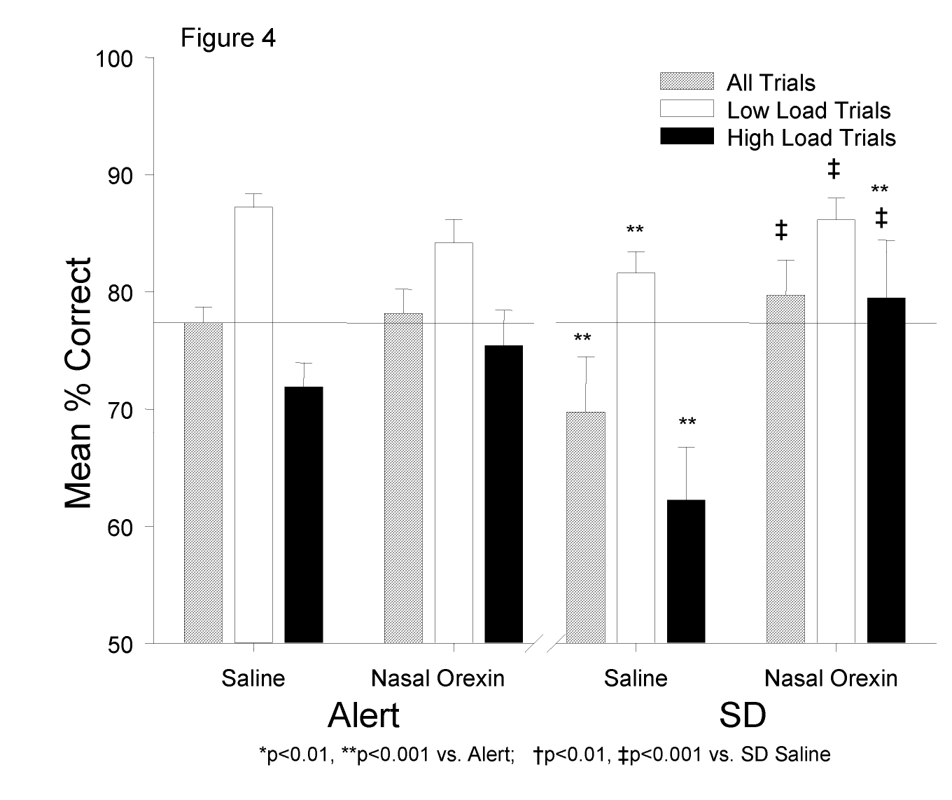

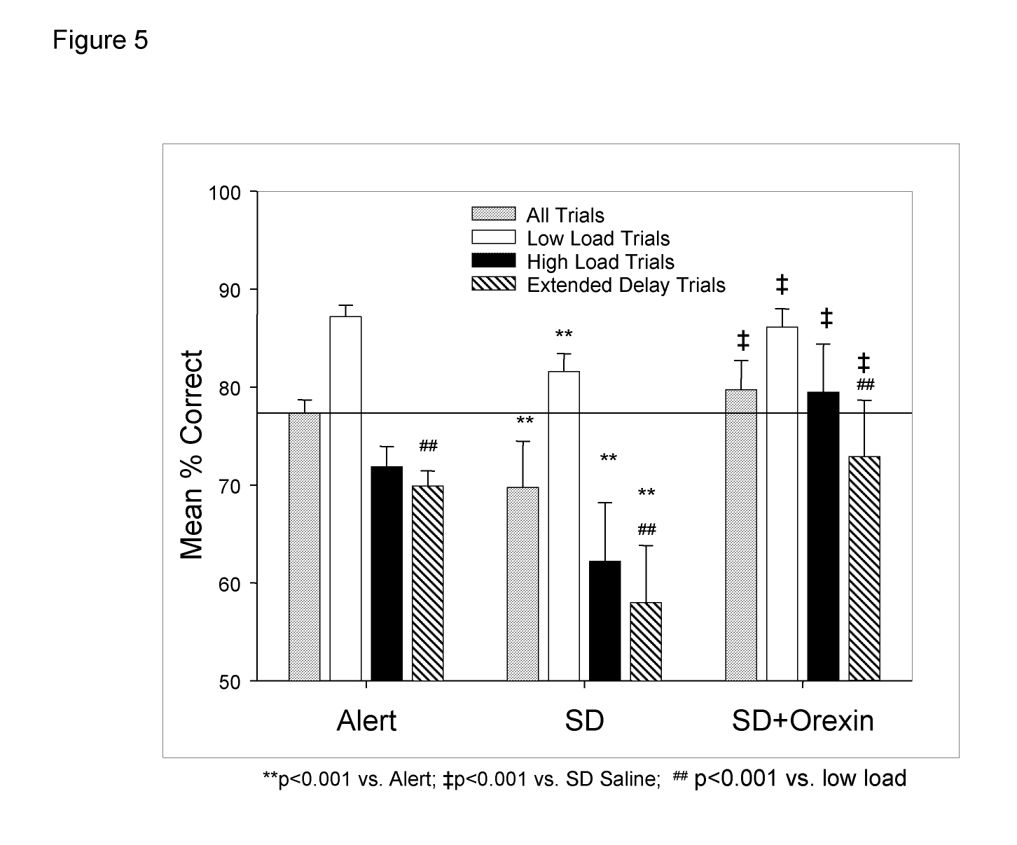

The effects of the nasal orexin-A spray in normal alert and sleep deprived sessions is shown in FIG. 4. On the left in FIG. 4 it is clear that nasal orexin-A had little or no effect on DMS performance in normal alert test sessions. Neither was performance affected on low and high cognitive load trials as was observed with IV orexin-A (FIG. 2). However, the surprisingly strong influence of nasal orexin-A on the performance of the same monkeys when sleep deprived is shown on the right in FIG. 4, and is in marked contrast to: 1) the effects of nasal orexin-A delivered in the same manner on normal alert sessions, and 2) the limited effectiveness of IV orexin-A administered to the same group of animals when sleep deprived (FIG. 2). The nasal orexin-A spray effectively reversed the detrimental influence of sleep deprivation on low cognitive load trials by returning performance to normal alert levels (F(_{1,503})=8.19, p<0.01 vs. SD Saline; F_{(1,503)}=1.14, N.S. vs. Alert Saline). More surprising however was the elevated performance above normal alert levels produced by nasal orexin-A on high cognitive load trials (F_{(1,503)}=9.07, p<0.001) which, compared to performance following control (saline) nasal spray in sleep deprived animals (F(_{1,503)}=17.53, p<0.001), was a considerable change (FIG. 4, right). To examine this latter effect in more detail performance was compared on trials with extended delays of 120 sec interleaved with regularly scheduled delays to further increase the difficulty (i.e. load) of high cognitive load trials. FIG. 5 shows the same data (FIG. 4) but with the extended delay trials added to indicate that performance on these type trials was also significantly above extended delay trials in both control sleep deprived and normal alert sessions (F_{(1,503)}>22.36, p<0.001). The latter finding further indicates that the impact of the nasal orexin-A was more substantial for difficult trials in sleep deprived animals than trials of low cognitive load (FIG. 4 and FIG. 5).

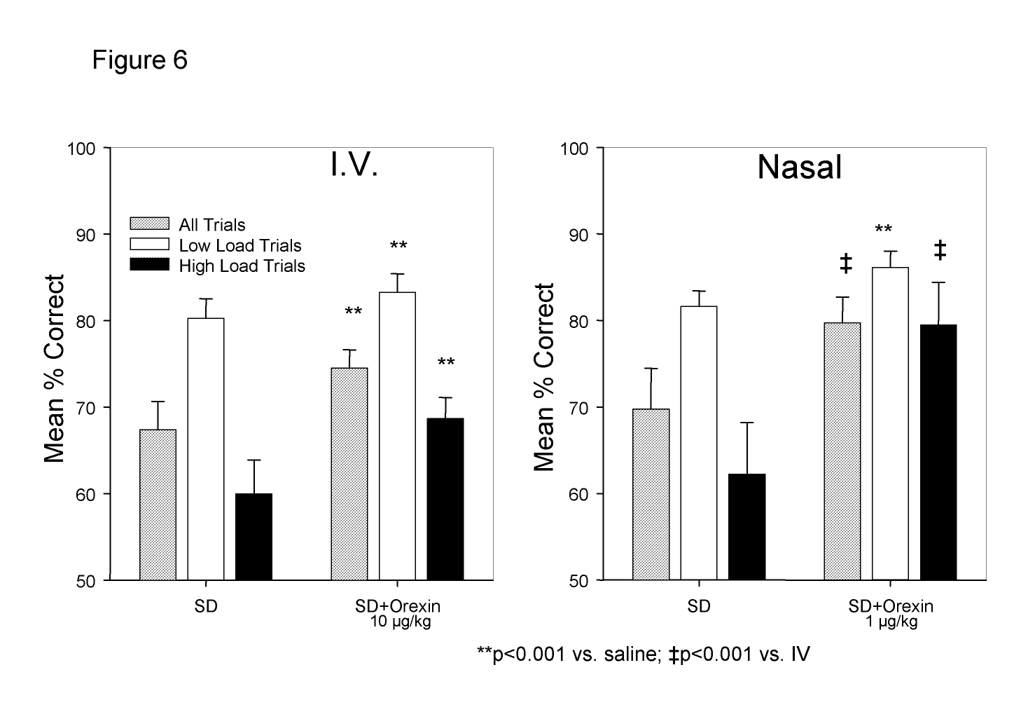

For purposes of comparison, performance under sleep deprived IV orexin-A and sleep deprived nasal orexin-A sessions are illustrated together in FIG. 6. There was no significant difference in baseline sleep deprived vehicle performance between the two test conditions, however it is clear that nasal orexin-A was significantly more effective overall (gray and on high cognitive load trials than the highest dose (10.0 μg/kg) of IV orexin-A (F_{(1,503)}=17.49, p<0.001). Thus nasal administration of orexin-A even though estimated to be significantly lower (1.0 μg/kg) in dose proved to be more potent than even the relatively high (10.0 μg/kg) dose of orexin-A delivered IV (Wu 2002) demonstrating that nasal delivery of orexin-A was not only a selective means of increasing performance on high cognitive load trials (i.e. there was no difference between IV and nasal orexin-A on low cognitive load trials), but the increase was substantial enough to return performance to above normal alert levels which was not achieved by the highest dose of IV orexin-A.

### Rates of Local Cerebral Metabolism During DMS Task Performance

Statistical parametric maps using SPM5 software (University College London) were generated for comparisons of relative CMRglc across the 8 different monkeys utilized in the above behavioral analyses of the effects of IV and nasal orexin-A. The isotope was injected immediately prior to each session and the animal anesthetized and ported to the PET scanner within 90 min to image 18F-flurodeoxyglucose uptake during performance of the DMS task We have replicated those effects.

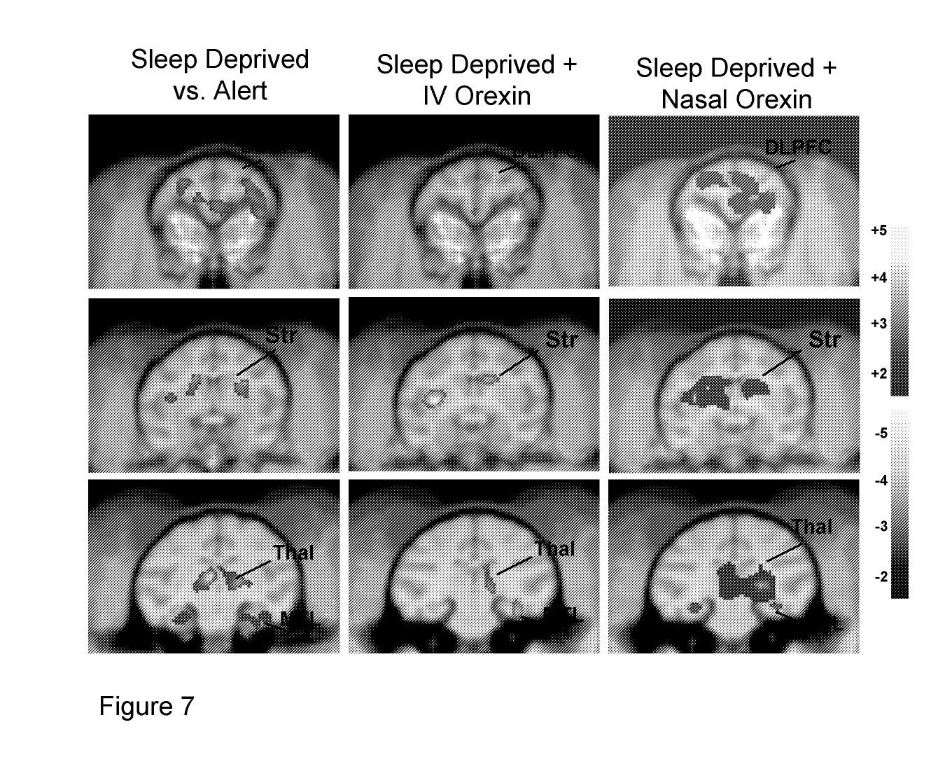

Effects of Sleep Deprivation on CMRglc: In a previous study we provided comprehensive analyses of changes in local CMRglc in specific brain regions associated with performance of the DMS task. In addition we showed how task-related local CMRglc in those regions is altered in monkeys that have been sleep deprived (Porrino, 2005). Consistent with that report, the current study showed that sleep deprivation produced both increases and decreases in relative CMRglc in specific brain regions engaged during performance of the DMS task. These changes are shown in FIG. 7 (left panel). Comparing CMRglc in the sleep deprivation condition to the normal alert task condition revealed significant decreases bilaterally in the dorsolateral prefrontal cortex (DPFC; left: t=7.75, p<0.001; right: t=4.61, p<0.01), and in the dorsal striatum (DStr; left: t=3.24; p<0.04; right: t=4.17. p<0.013). In contrast, marked increases in CMRglc in the medial temporal lobe (MTL; left: t=3.66, p<0.014; right: t=4.3, p<0.003) were obtained in the same comparison. In addition, a large decrease in CMRglc was observed in the thalamus (Thal; left: t=6.11, p<0.003; right: t=3.84, p<0.02) during performance of the task in the Sleep Deprivation condition.

Effects of IV orexin-A on CMRglc in Sleep Deprived Monkeys: Administration of the highest dose (10.0 μg/kg) of IV orexin-A to sleep deprived monkeys produced significant changes in CMRglc in each of the brain regions affected by sleep deprivation during performance of the task. Local CMRglc in the DPFC in the right hemisphere (FIG. 7, middle panel) was elevated by IV orexin-A in sleep deprived animals compared to levels that were decreased as shown previously (Porrino 2005) during the sleep deprivation condition (right: t=2.09, p<0.047). Similar increases from depressed sleep deprivation levels were observed bilaterally in the DStr (left, t=5.01, p<0.009, right, t=5.68, p<0.005) and Thal (right: t=2.82; p<0.03) in sleep deprivation sessions preceded by injections of IV orexin-A (10 μg/kg). Significant decreases in CMRglc were observed in the MTL (right: t=3.23, p<0.018) in the sleep deprived+IV orexin-A condition which constituted a directional change from previously reported (Porrino 2005) elevated levels in the sleep deprivation vehicle sessions (FIG. 7, middle bottom panel). Thus, DPFC, DStr, and Thal showed significant increases during task performance under sleep deprived conditions following IV orexin-A, while MTL showed a significant decrease, which (except for Thal), is consistent with previous findings showing changes in CMRglc associated with improvement in performance under sleep deprived conditions (Porrino 2005).

Effects of Nasal Orexin on CMRglc in Sleep Deprived Monkeys: The administration of nasal orexin-A as a spray mist (1.0 μg) to sleep deprived monkeys also produced significant changes in CMRglc in each of the four brain regions affected by sleep deprivation in the sessions in which performance was markedly facilitated (FIG. 4 and FIG. 5). CMRglc in the DPFC was significantly elevated bilaterally compared to sleep deprivation vehicle sessions (left: t=7.25, p<0.001; right: t=5.64, p<0.008). Similar increases were found bilaterally in DStr (left, t=9.33, p<0.001, right, t=4.48, p<0.02) and Thal (left: t=3.16, p<0.01; right: t=22.03; p<0.000). As with IV orexin-A, significant decreases in CMRglc were observed in the MTL (left, t=3.59, p<0.023) relative to the sleep deprivation alone baseline condition. Thus, the pattern of CMRglc changes in the DPFC, DStr, MTL and Thal (FIG. 7 right panel) in sleep deprived monkeys during DMS performance following administration of nasal orexin-A was similar to IV orexin-A, however the magnitude of the changes were markedly different and was consistent with the changes in performance shown in FIG. 6.

Comparison of Nasal vs. IV Administered orexin-A on CMRglc: To directly assess differences in the routes of administration of the scans obtained under IV orexin-A vs. nasal orexin-A for sleep deprivation sessions were compared in the same monkeys. Analyses of differences between the two delivery methods revealed that nasal orexin-A produced greater increases in CMRglc bilaterally in DPFC (left: t=3.54, p<0.05; right: t=7.18, p<0.003), DStr (left: t=3.10, p<0.05; right: t=3.39, p<0.033) Thal (left: 3.68, p<0.035; right: t=3.52, p<0.05) as well as decreased CMRglc in MTL (left: t=6.53, p<0.008; right: t=9.66, p<0.003), than IV orexin-A (not shown). However, no significant difference was detected between the two routes of administration with respect to magnitude of change in the DStr. Given the fact that both methods were compared in sleep deprived animals, the results of this comparison strongly suggests that the difference in potency for reversal of the effects of sleep deprivation in these brain regions was the basis for the corresponding difference in degree of improved performance under sleep deprived conditions shown in FIG. 6.

### Discussion

The above results show that: 1) orexin-A, administered either intravenously (IV) or via the nasal spray method employed here was a effective agent for reducing or reversing the effects of sleep deprivation on performance of the DMS task (FIGS. 2-4), 2) orexin-A delivered by both methods significantly reversed changes produced by sleep deprivation on local brain CMRglc during performance of the DMS task (FIG. 5), 3) the nasal route of administration of orexin-A was more effective with respect to both of these measures than if administered IV, and 4) neither IV nor nasal administered orexin-A produced facilitative effects if the animals were not sleep deprived or drowsy. Moreover, by completely reversing the effects of sleep deprivation on high cognitive load trials and showing larger reversals of local CMRglc levels in brain regions engaged by the DMS task, delivery of orexin-A via the nasal route was also likely more potent since the spray mist (estimated to be 1.0 μg) was only approximately 1/10th of the most effective IV dose (10.0 μg/kg). Another difference which cannot be accounted for at this point, is the slight but significant performance impairment of IV orexin-A at the higher dose range (10.0 mg/kg) which was not associated with nasal orexin-A delivered to alert animals.

Orexin-A (hypocretin-1) is a ubiquitous peptide found in all mammalian brains which has been extensively documented in anatomical studies showing localized populations of hypocretin containing cells in the hypothalamus and in areas lining the ventricular surface (Peyron 1998, van den Pol 1998, Kilduff, 2000, Moore 2001, Fadel 2002). In several animal models orexin-A administration has been shown to reverse the effects of sleep as well as increase performance in sleep deprived animals (Bourgin 2000, Piper 2000, Hagan 1999, Kiyashchenko 2001, Milekovskiy 2005). In addition several reports have demonstrated the correspondence between orexin-A release and the sleep-wake cycles of rodents, monkeys and humans (Edgar 1993, Chemellli 1999, Taheri 2000, Nishino 2000, Hara 2001, Yoshida 2001, Kiyashchenko 2002, Wu 2002, Zeitzer 2003, Lee 2005). Recently, the importance of this endogenous system for sleep cycle regulation was demonstrated by the use of the selective hypocretin/orexin receptor antagonist ACT-078573 (Actelion Pharmaceuticals) in rodents, dogs and man (Brisbare-Roch 2007).

The findings reported here support the above findings and showed that systemically administered (IV) orexin-A exhibited some efficacy at relatively high doses (10.0 μg/kg) in alleviating the deleterious effects of sleep deprivation on cognitive performance (FIG. 3 and FIG. 4). However, the relative cost and the lack of bioavailability of orexin-A coupled with drastically different potency when injected IV vs. intracerebrally (Kiyashchenko, 2001, Wu 2002, Vittoz, 2006) has precluded systemic orexin-A as a candidate for controlling the effects of sleep deprivation as well as other uses (Hara 2001). We show here that these limitations can be greatly overcome by delivering orexin-A via nasal spray which proved superior to systemically delivered orexin-A both in terms of enhancing performance in sleep deprived monkeys as well as reversing the effects on altered CMRglc levels in key brain areas affected by sleep deprivation.

The correspondence between the effects of nasal orexin-A in reversing the effects of sleep deprivation on DMS task performance and the concomitant differential reversal of the altered CMRglc levels in DFPC, DSt, Thal and MTL associated with those performance deficits, is consistent with our previous findings which utilized the ampakine CX717, a drug with completely different pharmacological actions on a different transmitter system to demonstrate similar effects (Porrino (2005)). The similarity in actions of the two compounds is supported by recent evidence showing that selective inactivation of orexin-A receptors in CA1 region of hippocampus impairs memory in rodents (Akbari 2006). Another basis for the facilitation in performance by orexin-A was the fact that unlike CX717, orexin-A administered by both routes nasal and IV, reversed the effects of sleep deprivation on CMRglc levels in the thalamus (Thal, FIG. 7), a major brain region highly susceptible to sleep deficits in humans (Thomas, 2000, Drummond, 2001, Lambe, 2005). The fact that nasal orexin-A was effective in reversing performance on high cognitive load trials, even to a greater extent even than on high cognitive load trials in normal alert sessions, suggests: 1) that high cognitive load trials engage either different brain processes or the same processes to a greater degree than low cognitive load trials, and 2) that “high load cognitive processes” may contain many neurons that have hypocretin-1 receptors that can be activated by the peptide in a preferential manner via the nasal delivery route (FIG. 6). Support for this assumption is provided by the large differences produced by nasal orexin-A on CMRglc levels in DFPC, Str and MTL compared to IV orexin-A administration. Another positive control for the specificity of this effect was the fact that no differences in CMRglc levels were observed in the thalamus as a function of the two delivery methods (Thal, FIG. 7).

Delivery of orexin-A via the nasal route and IV administration have been compared in terms of peptide binding to hypocretin-1 receptors in the CNS in anesthetized mice (Hanson 2004). Inhalation has been reported to be an effective means of delivery for a number of peptides, which cannot be delivered effectively in blood because of excessive protein binding (McFarland, 2002, Trombetta, 2005). However the effectiveness of nasal orexin-A delivered in this manner to all monkeys indicates that the nasal mucosa is a reliable means of penetration for the peptide to gain accessibility to important task-relevant brain processes whose CMRglc levels were completely reversed from the decreased levels produced by sleep deprivation. The involvement of neurons with hypocretin-1 receptors in the above process must await further investigation. Recent evidence showing that manipulation of hypocretin-1 binding via administration of the antagonist SB-334867 (Smart 2001, Rasmussen 2007) however, suggests that the effects observed here were specific to the hypocretin system since orexin-A administered by either route in non-sleep deprived animals was ineffective.

The current findings provide further evidence for the involvement of the sleep peptide hypocretin-1 (orexin-A) in the regulation of important cognitive processes that are affected by physiological perturbations such as sleep deprivation. The results demonstrate that orexin-A can reverse the cognitive performance deficits produced by sleep deprivation (FIG. 2 and FIG. 5). as well as reverse regionally specific CMRglc levels in task-related brain regions (FIG. 7). This suggests that orexin-A has effects that go beyond mere global arousal, and may be involved in reactivating specific brain mechanisms required for optimal performance in a task requiring both attention as well as working memory. The demonstration that orexin-A delivered via nasal spray in nonhuman primates is capable of reversing so many different behavioral and brain attributes affected by sleep deprivation provides a new basis for examining the actions of this and other brain peptides whose systemic delivery was heretofore not practical because of unknown interactions with constituents in blood and other absorption problems. Such investigation may reveal new opportunities to utilize orexin-A and other peptides in applications related to sleep, as well as other types brain disorders, via a much less invasive route of administration.

### Example 2

### Hyocretin Improves Cognitive Function in Drowsy Monkeys

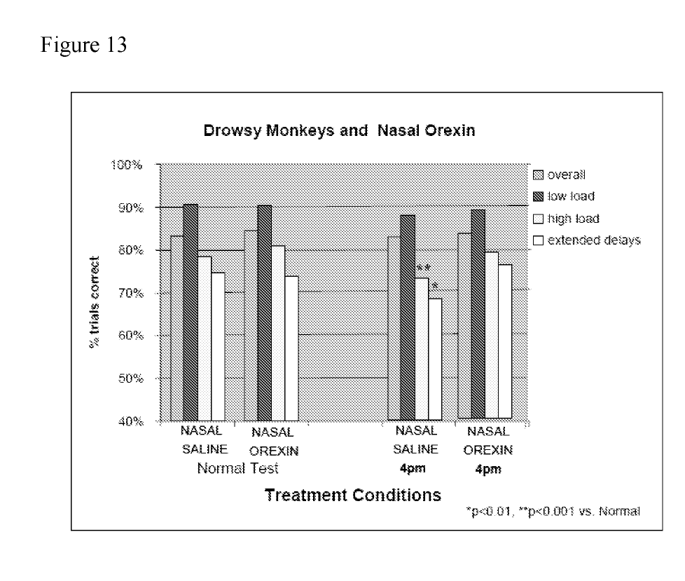

Monkeys were tested at the end of their normal day 12 hr day at 4 PM, approximately 6-8 hours later than their normal testing period, in what can be considered a “drowsy” condition just prior to the onset of their dark cycle at 6 PM (Price 2003, Thomas 2000). At this time animals were impaired in performance relative to their normal testing times (high cognitive load: F(1.72)=8.49. p<0.001; extended delays-very high cognitive load: F(1.72)=5.84, p<0.01) during the day. This effect was reversed completely (high load: F(1.72)=6.17. p<0.01; extended delay: F(1.72)=8.42, p<0.001) by administration of the sleep peptide hypocretin-1 (orexin-A) delivered via a nasal spray mist aimed at the nostrils of each monkey at a dose of 1.0 μg (25 microgram/ml in 0.04 ml of saline) per estimated spray. F refers to an analysis of variance and the numbers in parentheses are the degrees of freedom, essentially the number of subjects and conditions (−1). The F value is the an indication of the effect size as measured by the analysis of variance, which is translated into a probability (p) value. This is a standard test for significance. FIG. 13 left shows that treatment with orexinA in trials with different cognitive loads under normal testing conditions had no effect on performance assessed at normal time. FIG. 13 right shows the same animals tested at the end of the day at 4 PM immediately prior to onset of dark cycle (6 PM). Performance was decreased in animals administered nasal sprays of saline at this time of testing. Nasal orexin-A administered at 4 PM improved performance back to normal levels.

### Example 3

### Treatment of Parkinson's Disease

Parkinson's disease (PD) is preceded and accompanied by daytime sleep attacks, nocturnal insomnia, REM sleep behavior disorder, hallucinations and depression, symptoms which are frequently as troublesome as the motor symptoms of PD. All these symptoms are present in narcolepsy, which is linked to a selective loss of hypocretin (Hcrt) neurons. We find that PD is also characterized by a massive loss of Hcrt neurons. Thus, the narcolepsy-like symptoms of PD can have the same cause and treatment as the symptoms of narcolepsy. We also see a substantial loss of hypothalamic melanin concentrating hormone (MCH) neurons. The losses of Hcrt and MCH neurons are significantly correlated with the clinical stage of PD, not disease duration, whereas the loss of neuromelanin cells is significantly correlated only with disease duration.

### Introduction

Sleep disturbances with a prevalence that ranges from 74% to 98% [Mochizuki, 2006; Parkkinen, 2005] are major problems in Parkinson's disease (PD), often more disturbing than its motor symptoms. Most PD patients have daytime sleep attacks that resemble narcoleptic sleep attacks and that may be increased with the use of dopaminergic agonists, but also occur independently of these agents [Rye 2006; Savitt, 2006; Frucht, 2002; Frucht, 1999; Arnulf, 2006; Arnulf, 2002; Arnulf, 2000]. Many PD patients have REM sleep at sleep onse [Arnulf, 2002; Onofrj, 2003]. REM sleep behavior disorder is common in PD [Gagnon, 2002; Schenck, 1992] as are hallucinations, some of which have been found to be linked to REM sleep phenomena [Benbir, 2006; Arnulf, 2000]. Recent work has shown that the sleepiness complaints of PD typically precede the motor symptoms and intensify as the disease progresses [Abbott 2005; Dhawan, 2006]. All of the above symptoms are also characteristic of narcolepsy, suggesting that these symptoms of narcolepsy and PD may have a common cause.

Other symptoms that are common, but not universal in narcolepsy are also found in PD. Eighty percent of PD patients experience sleep fragmentation resulting from frequent and prolonged awakenings [Askenasy 2001]. This may be exacerbated by the movement disorders of PD but does not appear to be entirely the result of this symptom [Arnulf 2006; Grandas, 2004; Barone 2004; Priano, 2003; Stocchi, 1998]. The incidence of major depression is markedly elevated in PD. Other chronic diseases are not accompanied by a similar incidence of depression [Frosh 2006]. Disrupted nighttime sleep and depression are also common in narcolepsy [Aldrich 1998; Siegel 1999]. One element of narcolepsy that appears to be absent in PD is cataplexy.

Human narcolepsy is caused by a loss of hypocretin (Hcrt) neurons [Peyron, 2000; Thannickal, 2000b; Thannickal, 2003]. Measurement of Hcrt in the cerebrospinal fluid (CSF) of PD patients has produced inconsistent results. Some studies have reported abnormally low levels, whereas others have reported values in the normal range [Drouot, 2003; Overeem, 2002; Yasui, 2006; Mignot, 2002]. We have reported that Hcrt levels rise by as much as 100% when dogs or cats play, as compared to levels in quiet waking [Kiyashchenko, 2002; Wu, 2002]. These findings and other similar findings suggest that any reduction in Hcrt level in PD may be secondary to the reduced movement caused by PD, rather than resulting from primary pathology of the Hcrt system. It has been speculated that the loss of dopamine neurons may be responsible for the sleepiness symptoms of PD [Dzirasa 2006], but this does not appear to explain the early onset of these symptoms nor their striking similarity to those of narcolepsy. Only by examining the Hcrt system directly can we determine if Hcrt cells are damaged in PD.

### Materials and Methods

The hypothalamus of eleven PD (mean age 79±4) and five normal (mean age 77±3) brains was examined (Table 2). Brains were fixed in 10% buffered formalin containing 0.1 M phosphate buffer (pH=7.4). The hypothalamus was cut into 40 μm sections. Sections were immunostained for hypocretin (Hcrt-1), melanin concentrating hormone (MCH), alpha synuclein and glial fibrillary acidic protein (GFAP). The substantia nigra of ten PD brains and seven normal brains were used for the study of neuromelanin pigmented cell loss. The severity of Parkinson's disease was assessed using the Hoehn and Yahr scale [Hoehn, 2001]. The level of neuropathology was assessed using the Braak staging criteria [Braak, 2003]. Cell number, distribution and size were determined with stereology techniques on a one in eight series. All values are reported as mean S.E.M. Comparisons were made using the t-test.

TABLE 2
Clinical data of Parkinson's and control subjects, and characteristics of Hcrt and MCH cells

| | | | | Hcrt | | MCH | | Clin. | | | |
|---|---|---|---|---|---|---|---|---|---|---|---|
| | | | | cell | | cell | | Stage | Path. | | |
| | | | | size | No of | size | | (Hoehn | Stage | | |
| | | | No of | area | MCH | area | Clinical | and | (Braak., | Duration | |
| Subjects | Age | Sex | Hcrt cells | (μm²) | cells | (μm²) | diagnosis | Yahr,) | 2003) | (years) | Medications |
| Controls | | | | | | | | | | | |
| C-A | 61 | M | 65833 | 408.48 | 131616 | 409.15 | Pneumonia, | | | | |
| | | | | | | | testicular tumor | | | | |
| C-B | 78 | M | 68932 | 312.41 | 123332 | 407.95 | Non neuronal | | | | |
| C-C | 81 | F | 83330 | 355.67 | 138425 | 388.65 | Cerebrovascular | | | | |
| C-D | 85 | F | 79219 | 309.71 | 130840 | 346.69 | Cancer - | | | | |
| | | | | | | | adenocarcinoma | | | | |
| C-E | 82 | M | 80632 | 379.42 | 137325 | 436.34 | Cerebrovascular | | | | |
| C-F | 73 | F | only | | | | Breast cancer | | | | |
| | | | substantia | | | | | | | | |
| | | | nigra | | | | | | | | |
| C-G | 80 | M | only | | | | Cancer - renal | | | | |
| | | | substantia | | | | | | | | |
| | | | nigra | | | | | | | | |
| Parkinson's | | | | | | | | | | | |
| PD-A | 85 | F | 58124 | 340.07 | 120250 | 338.30 | PD | I | 3 | 20 | n/a- |
| PD-B | 68 | M | 59728 | 347.44 | 98924 | 336.85 | PD, dysphagia, | II | 4 | 23 | Sinemet, |
| | | | | | | | dementia | | | | bromocriptine, |
| | | | | | | | | | | | dopamine. |
| PD-C | 77 | M | 55505 | 318.68 | 110555 | 392.45 | PD, | II | 3 | 18.5 | bromocriptine, |
| | | | | | | | Alzheimer's | | | | Sinemet, |
| | | | | | | | | | | | Parlodel. |
| PD-D | 70 | F | 53374 | 347.39 | 79582 | 440.46 | PD | III | 3 | 4 | Sinemet |
| PD-E | 81 | M | 44266 | 306.61 | 69732 | 373.01 | PD | III | 4 | 13 | Sinemet, |
| | | | | | | | | | | | Parlodel |
| PD-F | 62 | M | 46800 | 304.49 | 71466 | 335.61 | PD | IV | 3 | 5 | n/a |
| PD-G | 90 | M | 45600 | 360.45 | 77600 | 413.50 | PD, | IV | 4 | 5 | Sinemet |
| | | | | | | | Alzheimer's | | | | |
| PD-H | 97 | F | 39642 | 297.49 | 62329 | 330.46 | PD, basilar | IV | 4 | 12 | Sinemet |
| | | | | | | | vasculature | | | | |
| PD-I | 62 | M | 29716 | 335.98 | 78571 | 341.44 | PD | IV | 4 | 9 | Sinemet, |
| | | | | | | | | | | | Permax |
| PD-J | 103 | F | 25866 | 342.41 | 32400 | 401.08 | PD | V | 6 | 21 | n/a |
| PD-K | 67 | M | 31742 | 345.47 | 40176 | 415.48 | PD, strokes | V | 5 | 19 | Sinemet, |
| | | | | | | | | | | | Eldepryl, |
| | | | | | | | | | | | Requip, |
| | | | | | | | | | | | Mirapex, |
| | | | | | | | | | | | Provigil |
| PD-L | 86 | F | only | | | | PD | II | n/a | 11 | Sinemet, |
| | | | substantia | | | | | | | | Permax, |
| | | | nigra | | | | | | | | Eldepryl |

Note:
Clin. = clinical,
Path. = pathological.

### Hcrt, MCH and Alpha Synuclein Immunohistochemistry

The sections were treated with 0.5% sodium borohydride in PBS for 30 min and washed with PBS, and then incubated for 30 min in 0.5% H₂O₂ for blocking of endogenous peroxidase activity. For antigen retrieval, sections were heated for 30 min at 80° C. in a water bath with 10 mM sodium citrate (pH 8.5) solution. The sections were cooled to room temperature in sodium citrate and washed with PBS. Water bath heating produces less tissue damage and more uniform antigen retrieval than other heating techniques (Jiao, 1999). After thorough washing with PBS the sections were placed for 2 hr in 1.5% normal goat serum in PBS and incubated for 72 hr at 4° C. with a 1:2000 dilution of Hcrt-1 (orexin-A, Calbiochem, San Diego, Calif.). Sections were then incubated in a secondary antibody (biotinylated goat anti-rabbit IgG; Vector Laboratories, Burlingame, Calif.) followed by avidin-biotin peroxidase (ABC Elite Kit; Vector laboratories), for 2 hr each at room temperature. The tissue-bound peroxidase was visualized by a diaminobenzidine reaction (Vector laboratories). Adjacent series of sections were immunostained for MCH (1:20000, polyclonal rabbit anti-melanin concentrating hormone, Phoenix Pharmaceuticals, Inc., Belmont, Calif.). Pretreatment and staining was carried out as described for Hcrt staining. Another series of one in twenty four sections with were used for α-synuclein staining (1:10000, mouse anti-alpha synuclein monoclonal antibody, Chemicon International, Temecula, Calif.). Sections were then incubated in a secondary antibody (biotinylated goat anti-mouse IgG; Vector Laboratories) followed by avidin-biotin peroxidase (ABC Elite Kit; Vector laboratories), for 2 hr each at room temperature. The tissue-bound peroxidase was visualized by a diaminobenzidine reaction (Vector laboratories).

### Double Immunolabelling

After antigen retrieval treatment, sections immunochemically stained for orexin and α-synuclein were incubated with a mixture of primary antibodies for orexin-A (1:2,000) and α-synuclein (1:10,000) for 72 hours at 4° C. After being rinsed, sections were sequentially incubated in biotinylated goat anti-mouse IgG (Vector Laboratories) for α-synuclein or biotinylated goat anti-rabbit IgG (Vector Laboratories) for orexin A and followed by avidin-biotin peroxidase (ABC Elite Kit; Vector laboratories) for two hours at room temperature. The final product of α-synuclein was visualized with nickel-DAB solution (Vector laboratories). The color of α-synuclein immunohistochemical products was black. The hypocretin immunohistochemical products were visualized with DAB, which had a yellow-brown color.

### GFAP Immunohistochemistry

For GFAP staining, sections were immunostained with a 1:2000 dilution of primary polyclonal rabbit anti-cow GFAP antibody (DAKO, Carpinteria, Calif.). Antigen retrieval was not required for GFAP staining. After a hydrogen peroxide treatment and blocking serum, the sections were immunostained with GFAP antibody followed by biotinylated goat anti-rabbit secondary antibody, and an avidin-biotin-HRP complex (Vectastain ABC kit, Vector laboratories). Incubation times were 24 hours (at 4° C.) for the primary antibody, 30 minutes (at room temperature) for the secondary antibody, and 1 hour (at room temperature) for the avidin-biotin-HRP complex. Sections were treated with the DAB reaction (Vector laboratories).

### Immunohistochemistry in Substantia Nigra

Substantia nigra of ten PD brains and seven neurologically normal brains were used (Table 2). The substantia nigra were cut into 40 μm thick coronal sections. Hematoxylin and eosin (FD Neurotechnologies Inc, Baltimore, Md.) staining were used for the identification of neuromelanin pigmented cells. A one in twenty four series of sections was stained for GFAP and alpha synuclein immunohistochemistry, with the same procedure used for the hypothalamic sections.

Control sections from each brain were processed without the primary antibody and did not show staining. Brain regions and nuclei were identified using the “Atlas of the Human Brain” (Mai, 2004). Digital image acquisition was carried out with a Micro Fire camera (Optronics, Goleta, Calif.) and imported to the Corel Draw program. Contrast and brightness were corrected.

### Quantitative Analysis

Hcrt and MCH cell number and distribution were determined with stereological techniques on a one in eight series of sections through the complete hypothalamus. We used a Nikon E600 microscope with three axis motorized stage, video camera, Neurolucida interface and Stereoinvestigator software (MicroBrightfield Corp., Colchester, Vt.). To find out whether alpha synuclein was colocalized with either Hcrt or MCH cells, we used Neurolucida mapping of the double immunolabelled sections.

The density of GFAP cells in the thalamus and posterior hypothalamus was calculated as the number of cells per unit area (mm²). After delineating the nucleus, we used 250×250 μm as the counting frame size for random sampling with stereological procedures. All values of each nucleus were calculated for each subject. These were pooled to give means and SEM for each region and each group.

To calculate the percentage loss of neuromelanin pigmented cells in the substantia nigra, we used Neurolucida mapping of each section stained with hematoxylin and eosin. The numbers of neuromelanin pigmented cells of PD brains were compared with matching sections of normals and the percentage loss was calculated.

The “nucleator probe” in the Stereology program was used to estimate the mean cross sectional area of the Hcrt, MCH and neuromelanin pigmented cells. Neurons with a clear nucleus were chosen for analysis. The nucleator probe was used with the optical fractionator and stereology procedures for systematic random sampling to identify cells (Gundersen, 1998). In the sampling results, the volume estimate associated with each cell was displayed, along with the average volume for the group of cells measured. A total of 606 Hcrt cells from normal (n=5) and 702 cells from PD (n=11) were measured. For MCH a total of 1032 (n=5) from normal and 1109 (n=11) from PD were measured. In the case neuromelanin pigmented cells, 1986 cells from normal (n=7) and 1518 cells from PD (n=10) were measured.

### Results

### Hcrt and MCH Cell Loss

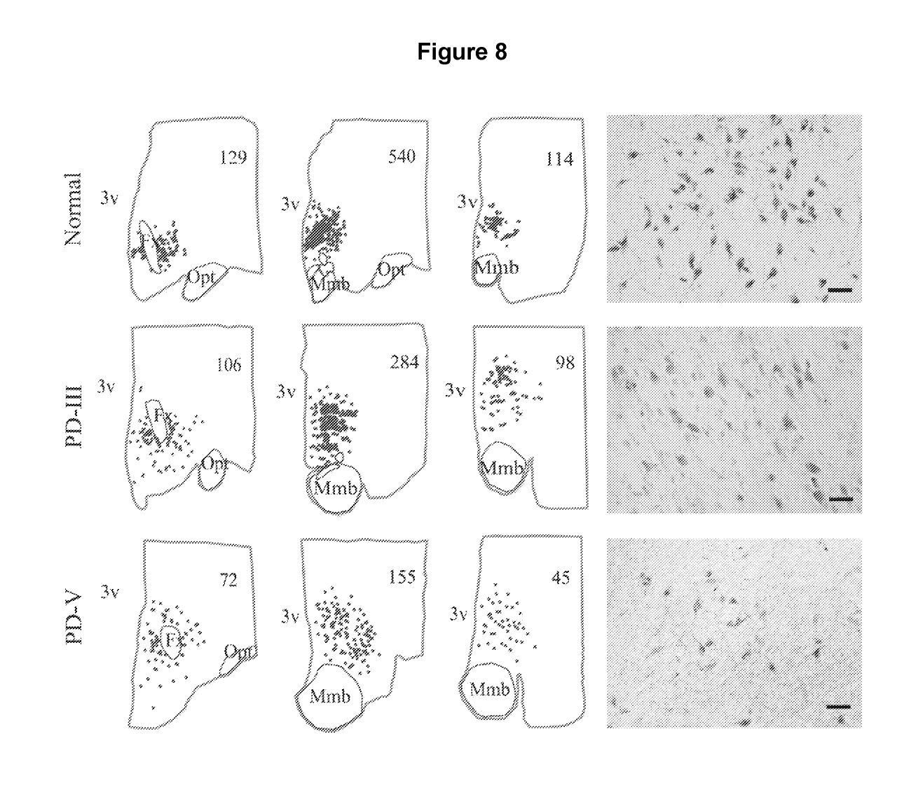

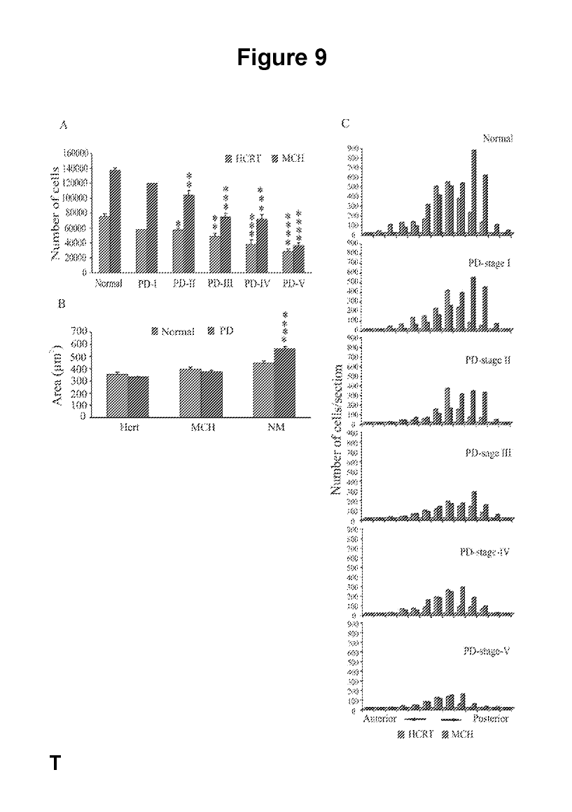

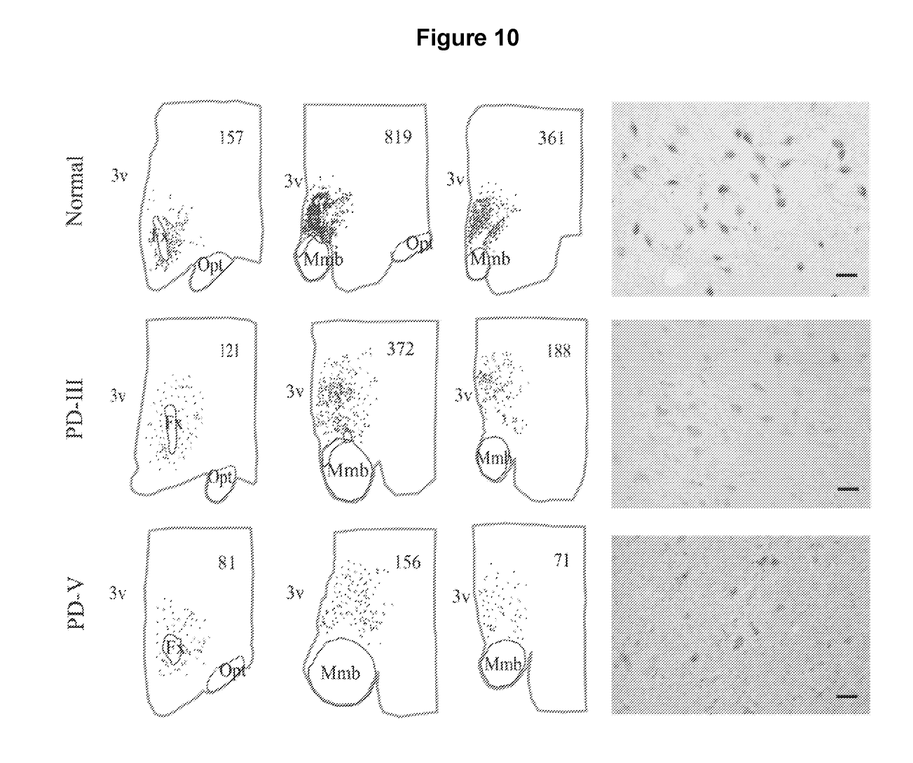

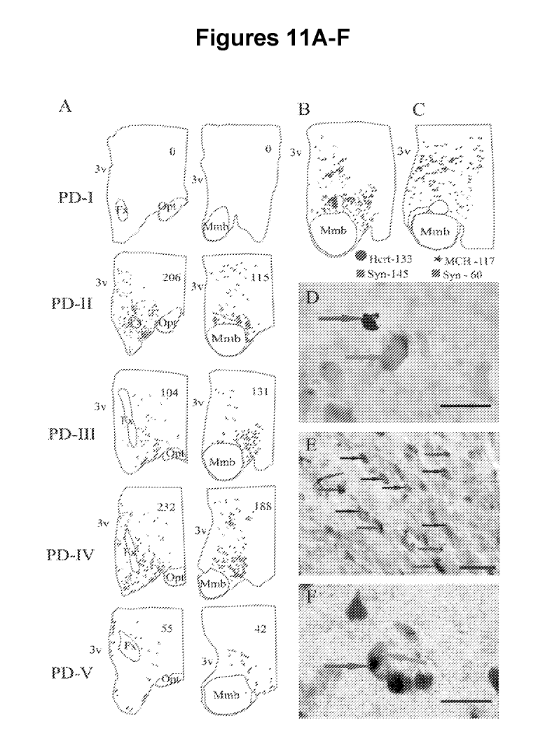

We found an increasing loss of hypocretin cells with disease progression (FIGS. 8 & 10) as measured by the Hoehn and Yahr rating scale (Hoehn, 2001). Similarly, there was higher loss of MCH cells with disease severity (FIGS. 9 & 10). Hcrt and MCH cells were lost throughout the A-P extent of their hypothalamic distributions (FIG. 10). The percentage loss of Hcrt cells was minimal in stage I (23%) and was maximal in stage V (62%). Similarly, the percentage loss of MCH cells was lowest in stage I (12%) and was highest in stage V (74%). There was a significant increase (p=0.0006, t=4.25, df=15) in the size of neuromelanin containing cells in PD patients as has been reported (Cabello, 2002), but no difference in the size of surviving Hcrt (p=0.18, t=1.39, df=14) and MCH (p=0.28, t=1.39, df=14) cells relative to control (FIG. 11B).

### Distribution of Alpha Synuclein, Gliosis and Neuromelanin Pigmented Cell Loss

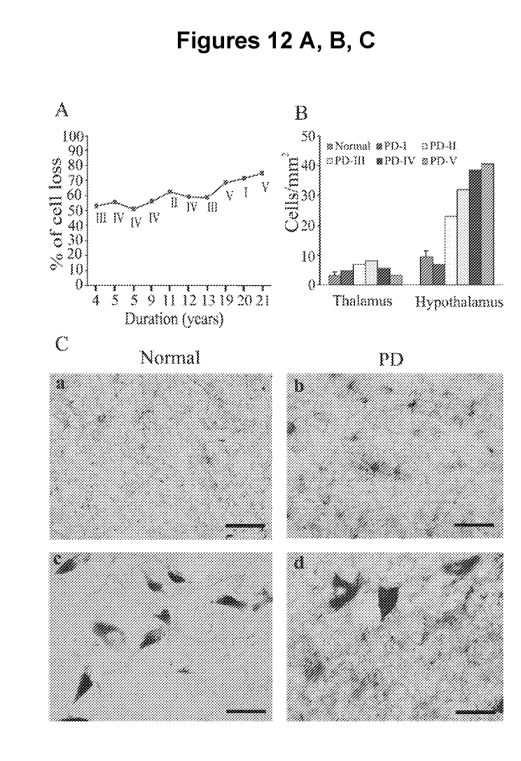

Alpha synuclein immunostaining showed a pattern of Lewy body formation in different stages of PD (FIG. 11A). We did not see Lewy bodies in surviving Hcrt (FIGS. 11B & D) or MCH cells (FIGS. 11C & E), but they were present in surviving neuromelanin containing cells of the substantia nigra (FIG. 11F). We hypothesize that these cells either die by a different mechanism than neuromelanin cells or that they die more rapidly, leaving few in an intermediate state to be observed. There was 50%-75% loss of neuromelanin pigmented cells in the substantia nigra (FIG. 12A) compared to control. In the hypothalamus, we saw increasing levels of GFAP with disease progression in PD (FIGS. 12B & C).

### Clinicopathological Correlations

We used the pathological variables (number of Hcrt, MCH and neuromelanin pigmented cells) and the clinical variables (severity and duration of disease) for the correlation study (Table 3). We found an increasing loss of hypocretin cells with disease progression as measured by the Hoehn and Yahr rating scale (Hoehn, 2001). Similarly, MCH cell loss was correlated with disease stage but not with disease duration In contrast, the loss of neuromelanin pigmented cells was not correlated with disease stage but was with disease duration, extending the conclusions of a recent study which showed that alpha synuclein pathology in neuromelanin cells does not correlate well with PD symptoms (Parkkinen, 2005). The Braak stages were correlated with percentage loss of neuromelanin pigmented cells, MCH, Hcrt, and the Hoehn & Yahr staging (Table 4).

TABLE 3
Correlation analysis of Hcrt, MCH and Neuromelanin
pigmented cell loss in PD with clinical stages
(Hoehn and Yahr), duration and % cell loss

| | | |
|---|---|---|
| Correlations | r | P |

% loss of cells versus PD stages

| | | |
|---|---|---|
| Hcrt cells and PD stages | 0.87 | 0.0005 |
| MCH cells and PD stages | 0.96 | 0.0001 |
| NM cells and PD stages | 0.03 | 0.94 |

% loss of cells versus PD duration

| | | |
|---|---|---|
| Hcrt cells and PD duration | 0.35 | 0.28 |
| MCH cells and PD duration | 0.04 | 0.90 |
| NM cells and PD duration | 0.92 | 0.002 |

% loss of cells versus % loss of cells

| | | |
|---|---|---|
| Hcrt cells and NM cells | 0.45 | 0.60 |
| MCH cells and NM cells | 0.25 | 0.58 |
| Hcrt cells and MCH cells | 0.83 | 0.001 |

Note:
r = correlation; P = significance; NM = neuromelanin pigmented cells

TABLE 4
Correlation analysis of pathological stages (Braak) in PD with
Hcrt, MCH and Neuromelanin pigmented cell loss and duration

| Correlations | r | P |
|---|---|---|
| % loss of Hcrt cells and pathological stages | 0.56 | 0.06 |
| % loss of MCH cells and pathological stages | 0.78 | 0.004 |
| % of NM cells and pathological stages | 0.86 | 0.001 |
| Duration and pathological stages | 0.53 | 0.09 |
| Duration and clinical stages | 0.09 | 0.78 |
| Pathological stages and clinical stages | 0.71 | 0.01 |

Note:
r = correlation; P = significance; NM = neuromelanin pigmented cells.

### Discussion

The early loss of Hcrt cells may be related to the early appearance of narcolepsy like signs in PD patients. This loss is occurring prior to the onset of drug treatment in many PD patients. The loss of Hcrt cells may also explain the orthostatic hypertension reported in PD [Hoehn, 2001] which parallels the low BP seen in Hcrt null mutant mice [Kayaba, 2003] and the abnormal regulation of body temperature that has been reported in both PD [Elliott, 1974] and Hcrt null mutant mice [Mochizuki, 2006].

The sleepiness experienced by PD patients may not be solely attributable to the loss of Hcrt neurons. It may be at lease partially due to the other neurodegenerative changes in PD, including the loss of dopamine, norepinephrine and serotonin neurons [Braak, 2003; Braak, 2004], all of which have alerting properties [Siegel 1990; Siegel 2005; Wisor, 2001; Aston-Jones, 2005]. The role of the loss of MCH cells reported here in the sleepiness of PD is unclear. In contrast to the maximal activity of Hcrt cells in waking [Lee, 2005; Mileykovskiy, 2005], MCH cells appear to be maximally active in sleep and are reciprocally connected with Hcrt neurons [Modirrousta, 2005; Alam, 2005; Torterolo, 2006; Verret, 2003]. The loss of MCH neurons in PD may therefore alter the expression of symptoms produced by loss of Hcrt neurons, which are selectively lost in narcolepsy. Loss of inhibitory input from MCH cells may maximize function in remaining Hcrt cells [Thannickal, 2000a], thereby preventing cataplexy.

If the loss of Hcrt cells is responsible for the symptoms common to both disorders, PD's narcoleptic like symptoms may respond to the same treatments found effective in narcolepsy. Especially promising would be treatment with hypocretin or hypocretin analogs [Siegel, 2006; John, 2003; John, 2000; Stocchi, 1998].

The significant correlations that we find between the loss of Hcrt and MCH neurons and the clinical stage of PD, in contrast to the lack of a relationship of similar strength between loss of neuromelanin containing cells and the clinical symptoms of PD, suggests a previously unappreciated relationship between hypothalamic dysfunction and the time course of the overall clinical picture of PD [Langston, 1978; Kremer 1993]. The demonstrated relation between Hcrt release and mood [Kiyashchenko, 2002; Mileykovskiy, 2005; Siegel 2004; Siegel, 2002] encourages the investigation of therapies targeted at reversing hypothalamic dysfunction to treat depression in PD.

### LIST OF REFERENCES

Abbott, Neurology 65, 442-1446 (2005).

Alam, J. Physiol. 563, 569-582 (2005).

Alberto, J. Neurosci. 26, 10043-10050 (2006).

Aldrich, Neurology 50, S2-7 (1998).

Akbari, Behay. Brain Res. 173, 47-52 (2006).

Arnulf, J. Neural. Transm. Suppl. 357-360 (2006).

Arnulf, Neurology 55, 281-288 (2000).

Arnulf, Neurology 58, 1019-1024 (2002).

Askenasy, Parkinsonism Relat. Disord. 8, 123-131 (2001).

Aston-Jones, J. Comp. Neurol. 493, 99-110 (2005).

Barone, Neurology, 63, S35-S38 (2004).

Baier, Brain (Aug. 21, 2008, Epub).

Benbir, Acta Neurol. Scand. 114, 239-243 (2006).

Berridge, Biol. Psychiatry 60, 111-20 (2006).

Borgland, Neuron 49, 589-601 (2006).

Bourgin, J Neurosci 20, 7760-7765 (2000).

Braak, Neurobiol Aging 24, 197-211 (2003).

Braak, Cell Tissue Res 318, 121-134 (2004).

Brisbare-Roch, Nat Med 13, 150-155 (2007).

Chemelli, Cell 98, 437-451 (1999).

Deutch, Schiz. Bull. 31, 510 (2005).

Dhawan, Age Ageing 35, 220-228 (2006).

Drouot, Neurology 61, 540-543 (2003).

Drummond, Neuropsychopharmacology 25, S68-S73 (2001).

Dzirasa, J. Neurosci. 26, 10577-10589 (2006).

Edgar, J. Neurosci. 13, 1065-1079 (1993).

Elliott, Neurology 24, 857-862 (1974).

Erny, Physiol. Behay. 35, 189-194 (1985).

Fadel, Neuroscience 111, 379-387 (2002).

Frosh, American Journal of Psychiatry 163, 1456-1457 (2006).

Frucht, JAMA 287, 2076-2077 (2002).

Frucht, Neurology 52, 1908-1910 (1999).

Gagnon, Neurology 59, 585-589 (2002).

Gerashchenko, J Neurosci 21, 7273-7283 (2001).

Grandas, Neurology 63, S8-11 (2004).

Hagan, Proc Natl Acad Sci USA 96, 10911-10916 (1999).

Hampson, Neuroreport 15, 1315-9 (2004)

Hanson, Drug Deliv Tech, 4, 1-10 (2004).

Hara, Neuron 30, 345-354 (2001).

Hervieu, Neuroscience 103, 777-797 (2001).

Hoehn, Neurology 57, S11-S26 (2001).

Horvath, J. Comp. Neurol. 415, 145-159 (1999).

Ishibashi, Peptides 26, 471-481 (2005).

Ishiia, Behav Brain. Res. 160, 11-24 (2005)

Ishiia, Behay. Brain. Res. 157, 331-341 (2005).

Jiao, Lancet 354, 427-8 (1999)

John , J. Physiol. (Lond.); 548.2, 557-562 (2003).

John, Sleep Res. Online 3, 23-28 (2000).

Kayaba, Am. J. Physiol. Regul. Integr. Comp. Physiol. 285, R581-R593 (2003).

Kennedy, Ann. Neurol. 4, 293-301 (1978).

Kilduff, Trends Neurosci. 23, 359-365 (2000).

Kiyashchenko, J. Neurophysiol. 85, 2008-2016 (2001).

Kiyashchenko, J. Neurosci. 22, 5282-5286 (2002).

Klerman, Sleep 22, 45-59 (1999).

Korotkova, J. Neurosci. 23, 7-11 (2003).

Kremer, Mov Disord 8, 315-320 (1993).

Lambe, J. Neurosci. 25, 5225-5229 (2005).

Langston, Ann. Neurol. 3, 129-133 (1978).

Lee , J. Neurosci. 25, 6716-6720 (2005).

Lin, Cell 98, 365-376 (1999).

McFarland, Med. Res. Rev. 22, 168-203 (2002).

Mellman, Adv. Exp. Med. Biol. 560, 63-67 (2005).

Mieda, Proc. Natl. Acad. Sci. USA 101, 4649-4654 (2004).

Mignot, Arch. Neurol. 59, 1553-1562 (2002).

Mileykovskiy, Neuron 46, 787-798 (2005).

Mochizuki, Am. J. Physiol. Regul. Integr. Comp. Physiol. 291, R533-R540 (2006).

Modirrousta, Eur. J. Neurosci. 21, 2807-2816 (2005).

Moore, Arch. Ital. Biol. 139, 195-205 (2001).

Murai, Neurosci. Res. 51, 55-65 (2005).

Onofr, Mov. Disord., 18, 337-340 (2003).

Overeem, Neurology February 12; 58 (3),498-9 (2002).

Nambu, Brain Res. 827, 243-260 (1999).

Nishino, Lancet 355, 39-40 (2000).

Parkkinen, Ann. Neurol. 57, 82-91 (2005).

Paxinos, The Rhesus Monkey Brain in Stereotaxic Coordinates. San Diego, Calif., Academic Press (2003).

Peever, J. Neurosci. 22, 5286-6 (2002).

Peyron, Nat. Med. 6, 991-997 (2000).

Peyron, J. Neurosci. 18, 9996-10015 (1998).

Priano, Neurol. Sci. 24, 207-208 (2003).

Phelps, Ann. Neurol. 6, 371-388 (1979).

Porrino, PLOS Biol. 3, e299 (2005).

Piper, Eur. J. Neurosci. 12, 726-730 (2000).

Price Anaesthesia 58, 1238-9 (2003)

Rasmussen, Neuropsychopharmacology 32, 786-792 (2007).

Rye, Curr. Neurol. Neurosci. Rep. 6, 169-176 (2006).

Savitt, J. Clin. Invest. 116, 1744-1754 (2006).

Schenck, Ann Neurol 32, 3-10 (1992).

Siegel, J. Clin. Neurophysiol. 7, 49-65 (1990).

Siegel, Narcolepsy Cell 98, 409-412 (1999).

Siegel, Annual Rev. Psychol. 55, 125-148 (2004).

Siegel In, Kryger, editor. Principles and Practice of Sleep Medicine. Philadelphia, Elsevier Saunders; p. 120-35 (2005).

Siegel, Nat. Clin. Pract. Neurol. 2, 548-556 (2006).

Siegel, J. Clin. Psychiatry 65 Suppl 16, 4-7 (2004).

Smart, Br. J. Pharmacol. 132, 1179-1182 (2001).

Smith, Neurosci. Lett. 341, 256-258 (2003).

Soffin, Neuropharmacology 42, 127-133 (2002).

Sokoloff, J. Neurochem. 28, 897-916 (1977).

Stocchi, J. Neurol. 245 Suppl 1, S15-S18 (1998).

Taheri, Neurosci. Lett. 279, 109-112 (2000).

Thannickal, Neuron 27, 469-474 (2000).

Thannickal, Abstr. Soc. Neurosci. a; 26, 2061 (2000).

Thannickal, Brain Pathol. 13, 340-351 (2003).

Thomas, J. Sleep. Res. 9, 335-352 (2000).

Torterolo, Brain Res, 1119, 101-114 (2006).

Trombetta, Annu. Rev. Immunol. 23, 975-1028 (2005).

van den Pol, J. Neurosci. 18, 7962-7971 (1998).

van den Pol, J. Neurosci. 19, 3171-3182 (1999).

Verret, BMC Neurosci. 4, 19 (2003).

Vittoz, Neuropsychopharmacology 31, 384-395 (2006).

White, Peptides 26, 2331-2338 (2005).

Wisor, J. Neurosci. 21 (5), 1787-94 (2001).

Woods, J. Comput. Assist. Tomogr. 17, 536-546 (1993).

Wu, Am. J. Physiol. Regul. Integr. Comp. Physiol. 283, R1079-R1086 (2002).

Yasui, J. Neurol. Sci. (2006).

Yoshida, Eur. J. Neurosci. 14, 1075-1081 (2001).

Zeitzer, J. Neurosci. 23, 3555-3560 (2003).

1. A method of treating at least one symptom of sleep deprivation in a subject in need thereof, comprising administering a hypocretin agonist to said subject in an amount effective to treat said at least one symptom of sleep deprivation.

2. The method of claim 1, wherein said agonist is orexin-A, an analog thereof, a prodrug thereof, or a pharmaceutically acceptable salt of any thereof, in an amount effective to treat or prevent said at least one symptom of sleep deprivation.

3. (canceled)

4. The method of claim 3, wherein the intranasal administration is by a nasal spray.

5. The method of claim 1, wherein the administration is performed within an hour before performing or during performance of a task whose performance would otherwise be impaired by the sleep deprivation.

6. The method of claim 5, wherein the task requires a high cognitive load.

7. (canceled)

8. The method of claim 1, wherein the agonist is orexin-A and the dose is 0.1-2 μg.

9. The method of claim 1, wherein the administration is performed at irregular intervals responsive to the patient performing tasks whose performance would otherwise be impaired by the sleep deprivation.

10. The method of claim 1, wherein the administration is performed multiple times during a continuous wake phase.

11. The method of claim 1, wherein the subject lacks a diagnosed sleep disorder or known biochemical or genetic marker of a sleep disorder.

12. The method of claim 1, wherein the subject is free of narcolepsy, REM sleep behavior disorder, period leg movements in sleep and restless leg syndrome, circadian rhythm disorder, sleep apnea, hypersomnia, insomnia, Alzheimer's disease, depression, schizophrenia, and obesity, and cataxplexy and is not in need of consolidation of sleep and waking states.

13-14. (canceled)

15. The method of claim 1, wherein the administration does not promote greater consolidation of sleep and waking states in the patient.

16. (canceled)

17. The method of claim 1, wherein the administration occurs after at least 18 hours of a wake phase.

18. The method of claim 1, wherein the administration occurs after at least 24 hours of a wake phase.

19-20. (canceled)

21. The method of claim 1, wherein said symptom comprises cognitive impairment.

22. The method of claim 21, wherein the administration improves the cognitive performance of the subject above a level without sleep deprivation.

23. The method of claim 1, wherein said subject is human.

24. (canceled)

25. The method of claim 1, further comprising concurrently administering at least one additional active compound for treating at least one symptom of sleep deprivation to said subject.

26. The method of claim 25, wherein said additional active compound is selected from the group consisting of: caffeine, nicotine, amphetamines, Modafinil, AMPA receptor potentiators and CX717.

27-41. (canceled)

42. A method of treating a symptom of Parkinson's disease comprising administering hypocretin or a hypocretin analog to a patient having Parkinson's disease.

43-47. (canceled)

48. A method of determining a clinical stage of Parkinson's disease in a patient, comprising:
determining a level of hypocretin in a body fluid of the patient, wherein loss of hypocretin is correlated with clinical stage of the disease.
